(19) United States
(12) Patent Application Publication
(10) Pub. No.: US 2019/0217293 A1
(43) Pub. Date: Jul. 18, 2019

(54) MICROFLUIDIC PLATFORM FOR THE CONCENTRATION AND DETECTION OF BACTERIAL POPULATIONS IN LIQUID

(71) Applicant: Tokitae LLC, Bellevue, WA (US)

(72) Inventors: Luis F. Alonzo, Tacoma, WA (US); Spencer Garing, Seattle, WA (US); Anne-Laure M. Le Ny, Issaquah, WA (US); Kevin Paul Flood Nichols, Issaquah, WA (US); Sam Rasmussen Nugen, Ithaca, NY (US); John R. Williford, Sammamish, WA (US)

(73) Assignee: Cornell University, Ithaca, NY (US)

(21) Appl. No.: 15/870,370

(22) Filed: Jan. 12, 2018

(51) Int. Cl. B01L 3/00; B01D 39/18; G01N 1/40; C12Q 1/04

(52) U.S. Cl. CPC B01L 3/502753; B01D 39/18; B01L 3/502738; B01L 2300/0681; C12Q 1/04; B01D 2239/1216; G01N 2001/4088; G01N 1/4077

(57) ABSTRACT

A microfluidic device for concentrating and detecting bacteria in liquids, and related methods are described. The device includes a first filter chamber for capturing bacteria and performing incubations of the bacteria with one or more reagents, and a second filter chamber for capturing and concentrating a detectable material, with little or no binding of detectable material by the first filter. In an aspect, bacteria are incubated with growth media and engineered phage that cause the bacteria to produce an enzyme. In an aspect, the enzyme is capture in the second filter chamber and exposed to a substrate to produce a detectable signal.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### Priority Applications

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a microfluidic device includes, but is not limited to, a sample inlet port adapted to receive a fluid sample containing bacteria of interest; a first filter chamber located downstream from the sample inlet port, the first filter chamber containing a first filter having a first area and formed from a first porous material having a pore size adapted to capture the bacteria of interest; a sample inlet channel connecting the sample inlet port to an upstream end of the first filter chamber; a sample control valve in the sample inlet channel, the sample control valve adapted to control a flow of the sample fluid from the sample inlet port to the upstream end of the first filter chamber; at least one first reagent inlet port located upstream of the first filter chamber and in fluid communication with the upstream end of the first filter chamber, the at least one first reagent inlet port adapted to deliver to the first filter chamber a first reagent containing a bacteriophage specific to the bacteria of interest and adapted to cause the bacteria of interest to release a reporter enzyme; at least one first reagent control valve adapted to control a flow of the first reagent from the first reagent inlet port to the upstream end of the first filter chamber; and a second filter chamber located downstream from the first filter chamber, the second filter chamber containing a second filter having a second area and formed from a second porous material adapted to specifically bind the reporter enzyme, wherein the second area is smaller than the first area; and a detection chamber control valve located downstream of the first filter chamber and adapted to control a flow of fluid to the second filter chamber; wherein the first filter is adapted to not bind the reporter enzyme. In addition to the foregoing, other aspects are described in the claims, drawings, and text forming a part of the disclosure set forth herein.

In an aspect, a method of concentrating bacteria for detection includes, but is not limited to, introducing a fluid sample containing bacteria of interest in a carrier fluid to a sample inlet port of a microfluidic device; drawing the carrier fluid through a first filter in a first filter chamber of the microfluidic device and through a waste port downstream of the first filter chamber while the bacteria of interest are captured by the first filter; drawing a first reagent including growth media for the bacteria of interest from a first reagent inlet port into the first filter chamber; incubating the bacteria of interest captured by the first filter with the first reagent in the first filter chamber for a first incubation period sufficient to increase at least one of the metabolic activity or the number of cells of the bacteria of interest; drawing the first reagent through the first filter and through the waste port while the bacteria of interest remain captured by the first filter; drawing a second reagent including a bacteriophage specific to the bacteria of interest from a second reagent inlet port into the first filter chamber; incubating the bacteria of interest captured by the first filter with the second reagent in the first filter chamber for a second incubation period sufficient to produce expression of a reporter enzyme by the bacteria of interest; drawing a fluid containing the expressed reporter enzyme through the first filter, through a second filter in a second filter chamber of the microfluidic device, and through the waste port while the expressed reporter enzyme is captured by the second filter; and incubating the expressed reporter enzyme captured by the second filter with a third reagent in the second filter chamber for a third incubation period sufficient to produce a detectable signal in the detection chamber. In addition to the foregoing, other method aspects are described in the claims, drawings, and text forming a part of the disclosure set forth herein.

In an aspect, a microfluidic device for bacteria detection includes, but is not limited to, a sample inlet port for receiving a fluid sample containing bacteria of interest; a first filter chamber containing a first filter adapted for capturing bacteria of interest from the fluid sample; first microfluidic means for introducing bacterial growth media to the first filter chamber; second microfluidic means for introducing phage specific to the bacteria of interest to the first filter chamber, the phage adapted to cause the bacteria of interest to produce a reactive material capable of reacting to produce a detectable signal; third microfluidic means for flushing reactive material from the first filter chamber, the reactive material released from the bacteria of interest responsive to introduction of the phage; and a second filter chamber containing a second filter for specifically capturing the reactive material flushed from the first filter chamber, wherein the second filter is smaller than the first filter to amplify the detectable signal; wherein the first filter is adapted to not capture the reactive material. In addition to the foregoing, other aspects are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A-1H illustrate a process for concentrating and detecting bacteria.

FIG. 2 is a schematic of microfluidic circuit.

FIG. 3 is a flow diagram of a method of concentrating bacteria for detection.

FIG. 4 is a flow diagram showing further aspects of the method of FIG. 3.

FIG. 5 is a flow diagram showing further aspects of the method of FIG. 3.

FIG. 6 is a flow diagram showing further aspects of the method of FIG. 3.

FIG. 7 is a flow diagram showing further aspects of the method of FIG. 3.

FIG. 8 is a flow diagram showing further aspects of the method of FIG. 3.

FIG. 9 is a flow diagram showing further aspects of the method of FIG. 3.

FIG. 10 depicts operation of the microfluidic circuitry of FIG. 2.

FIG. 11 depicts operation of the microfluidic circuitry of FIG. 2.

FIG. 12 depicts operation of the microfluidic circuitry of FIG. 2.

FIG. 13 depicts operation of the microfluidic circuitry of FIG. 2.

FIG. 14 depicts operation of the microfluidic circuitry of FIG. 2.

FIG. 15 depicts operation of the microfluidic circuitry of FIG. 2.

FIG. 16 depicts operation of the microfluidic circuitry of FIG. 2.

FIG. 17 depicts operation of the microfluidic circuitry of FIG. 2.

FIG. 18 is a top view photo of a microfluidic device.

FIG. 19A is a cross-sectional diagram of a filter chamber taken at section line A-A in FIG. 18.

FIG. 19B is a cross-sectional diagram of a filter chamber taken at section line B-B in FIG. 18.

FIG. 20 is a top view of an alternative microfluidic device design.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present invention relates to methods and system for detecting the presence of contaminants such as bacteria in liquids. In particular, the present invention relates to microfluidic devices for concentration and detection of bacteria in liquids.

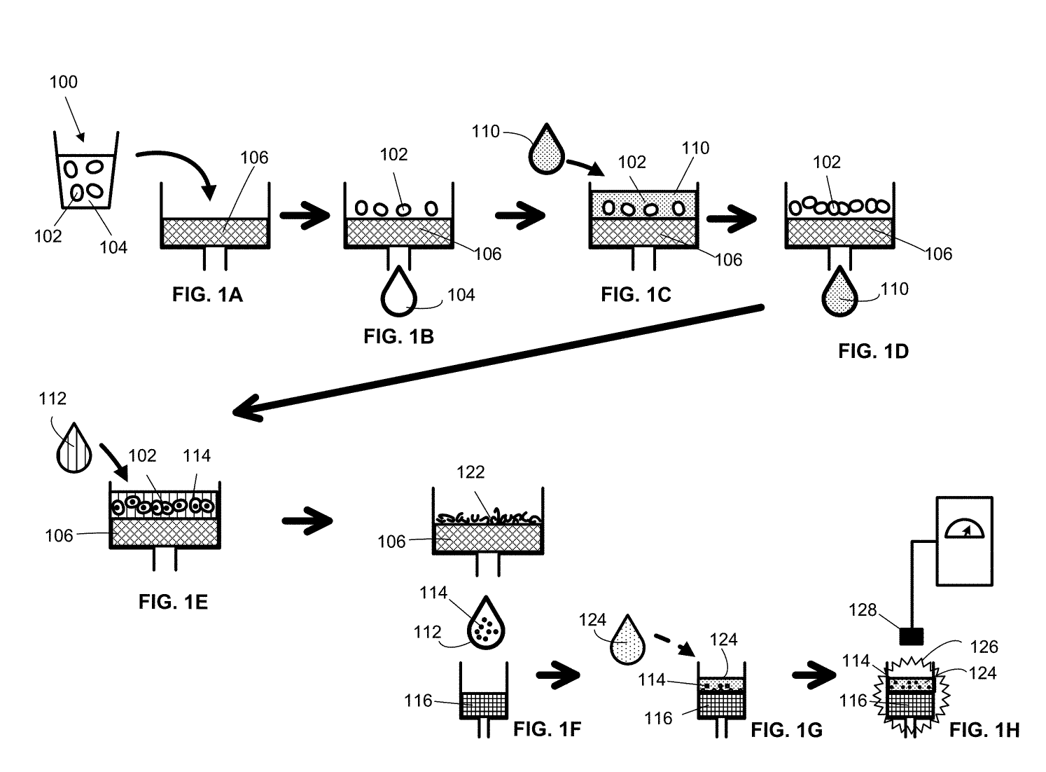

FIGS. 1A to 1H illustrate in simplified form a process for concentrating and detecting bacteria, suitable for performance in a microfluidic device. In FIG. 1A, a sample 100 containing bacteria 102 in fluid 104 is added to a first filter 106. For example, it is of interest to detect the presence of Escherichia coli (E. coli) in drinking water. In FIG. 1B, fluid 104 passes through first filter 106, while bacteria 102 are captured by first filter 106. In FIG. 1C, growth media 110 are added, and bacteria 102 are incubated in growth media 110 on first filter 106, during a first incubation. In an aspect, bacteria present in an environmental sample are in a stationary growth phase. During the first incubation, the metabolic rate of the bacteria increases as the bacteria are exposed to growth media. Recovery of metabolic rate may take about 2 hours, for example. In an aspect, bacteria are allowed to replicate following metabolic recovery, to increase their numbers. For example, in cases where low bacterial concentrations are expected, bacteria may be allowed to replicate to produce a larger detectable signal. Bacterial replication can be obtained by incubating the bacteria in growth media for a sufficiently long amount of time after their metabolic rate has recovered (e.g., depending on the type of bacteria, about 20 minutes may be enough time for the bacterial population to double after metabolic rate has recovered). In FIG. 1D, growth media 110 are removed from first filter 106, while bacteria 102 are captured by filter 106. In FIG. 1E, a reagent 112 containing an engineered phage is added to first filter 106. The engineered phage causes bacteria 102 to produce an enzyme 114 as well as replicate the phage. In an aspect, lytic protein released by the phage causes lysis of the bacteria, releasing phage and enzyme during a second incubation. In FIG. 1F, following the second incubation, enzyme 114 is flushed through first filter 106 to second filter 116, carried by reagent 112. Additional fluid (e.g. an additional wash of growth media) may be used to ensure complete transfer. Lysed bacteria 122 remain in first filter 106. As shown in FIG. 1G, during a third incubation, enzyme 114 captured in second filter 116 is incubated with an enzyme substrate 124. In an aspect, enzyme substrate 124 is added to the second filter 116 just prior to the third incubation. Following the third incubation, as shown in FIG. 1H, a detectable signal 126 produced by reaction of enzyme 114 with enzyme substrate 124 is detected from second filter 116 with a detector 128.

Important aspects of the process illustrated in FIGS. 1A to 1H are that first filter 106 captures the bacteria 102, but not enzyme 114, and that second filter 116 captures enzyme 114. First filter 106 captures and concentrates bacteria 102 from liquid sample 100. Second filter 116 has a smaller area than first filter 106, in order to concentrate enzyme 114 to produce a greater detectable signal 126. In an aspect, the “area” of the first filter or the second filter is a “binding area” or “effective filtering area” of the filter, which is related to the surface area of the filter but is not necessarily identical to the surface area of the filter. The first filter and the second filter are independently optimized for their respective functions.

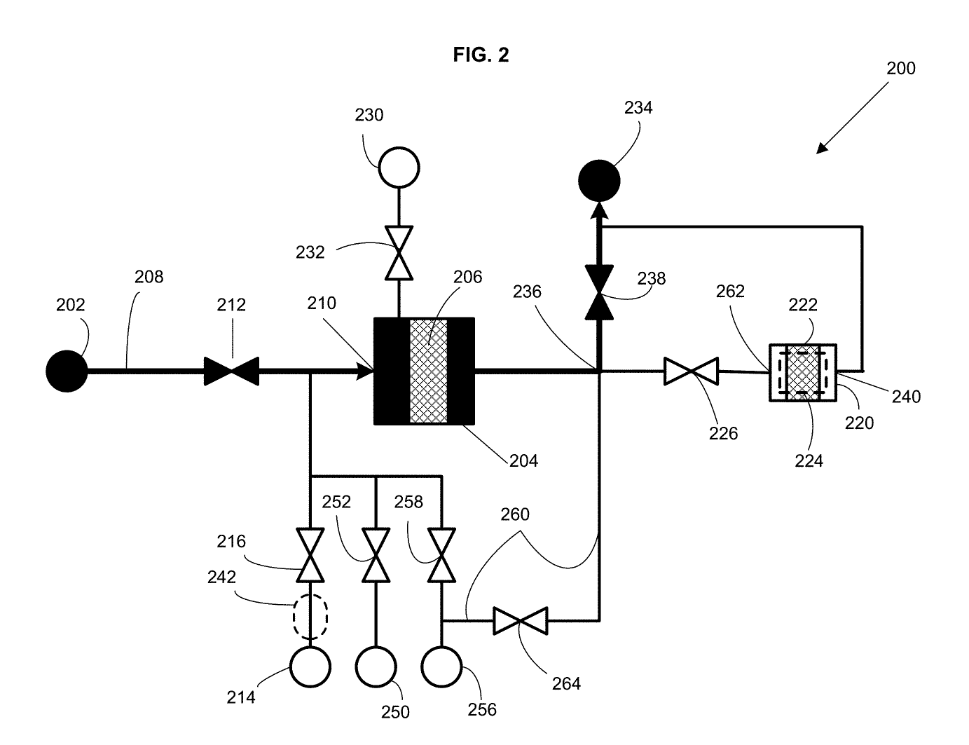

FIG. 2 is a schematic diagram of a microfluidic device 200 for performing a process as outlined in FIGS. 1A-1H. Microfluidic device 200 includes a sample inlet port 202 adapted to receive a fluid sample containing bacteria of interest, and a first filter chamber 204 located downstream from the sample inlet port 202. For example, in an aspect, microfluidic device 200 is adapted to process a fluid sample having a volume of at least about 100 ml. First filter chamber 204 contains a first filter 206 having a first area and formed from a first porous material having a pore size adapted to capture the bacteria of interest. For example, in an aspect the first porous material has a pore size of about 0.45 μm. In an aspect, the first porous material has a pore size of less than about 0.45 μm. In an aspect, the first filter functions to filter bacteria from the sample fluid, which may be, for example, an environmental sample. In an aspect, the first porous material is a non-cellulose material. For example, in various aspects, the first porous material is formed from polyvinyilidene fluoride (PVDF), polycarbonate (PC), tracked-etched polycarbonate (PCTE), polyethersulfone (PES), and tracked-etched polyester. Use of non-cellulose material in the first filter prevents or minimizes binding of reporter enzyme to the first filter when the reporter enzyme includes a cellulose binding region (as discussed elsewhere herein). In general, the first filter material is selected such it captures the bacteria of interest without significantly binding the reporter enzyme (or other reporter molecules or materials). In an aspect, the first porous material has low protein binding activity.

Sample inlet channel 208 connects sample inlet port 202 to an upstream end 210 of first filter chamber 204, and sample control valve 212 in sample inlet channel 208 is adapted to control a flow of sample fluid from sample inlet port 202 to upstream end 210 of first filter chamber 204. Microfluidic device 200 includes at least one first reagent inlet port 214 located upstream of first filter chamber 204 and in fluid communication with the upstream end 210 of first filter chamber 204. First reagent inlet port 214 is adapted to deliver to first filter chamber 204 a first reagent containing a bacteriophage specific to the bacteria of interest and adapted to cause the bacteria of interest to release a reporter enzyme. First filter 206 is adapted to bind the bacteria of interest, but not bind the reporter enzyme. At least one first reagent control valve 216 is adapted to control a flow of the first reagent from first reagent inlet port 214 to the upstream end 210 of first filter chamber 204. A second filter chamber 220, which functions as a detection chamber (from which a detectable signal can be detected) is located downstream from first filter chamber 204. Second filter chamber 220 contains a second filter 222 having a second area and formed from a second porous material adapted to specifically bind the reporter enzyme. In an aspect, the second area is smaller than the first area. For example, in an aspect, the first area is about 315 mm² and the second area is about 3.14 mm².

The function of the second membrane is to capture the enzyme, which in an aspect contains a cellulose binding domain. Accordingly, the second porous material includes a cellulose-based material such as regenerated cellulose, cellulose acetate, cellulose ester, and nitrocellulose. The size of the membrane is selected to concentrate the chemiluminescence reaction onto a smaller surface area for increased output signal.

In an aspect, the second porous material has a pore size of about 0.2 μm, for example. However, cellulose based porous materials are available with a variety of pore sizes, and materials with other pore sizes may be used, as appropriate for specific applications. In an aspect, second filter chamber 220 includes a detection region 224 configured to allow detection of a signal resulting from the reporter enzyme from outside the microfluidic device. In an aspect, detection region 224 includes a window formed from a clear material in microfluidic device 200, allowing a signal resulting from reaction of the reporter enzyme with an enzyme substrate to be detected from outside microfluidic device 200.

A detection chamber control valve 226 is located downstream of first filter chamber 204 and adapted to control a flow of fluid to second filter chamber 220.

In general, fluid channels connecting components of microfluidic device 200 have dimensions on the order of a 100 μm high and a millimeter or two wide. For example, in various aspects, two or more of sample inlet port 202, the at least one first reagent inlet port 214, first filter chamber 204, and second filter chamber 220 are fluidically connected by at least one fluid channel having a width of about 2 mm and height of about 100 μm. In some aspects, fluid channels may be between about 1 mm wide and about 3 mm wide and up to about 200 μm high. Different channel geometries may be used, depending upon the volume and types of fluids being handled.

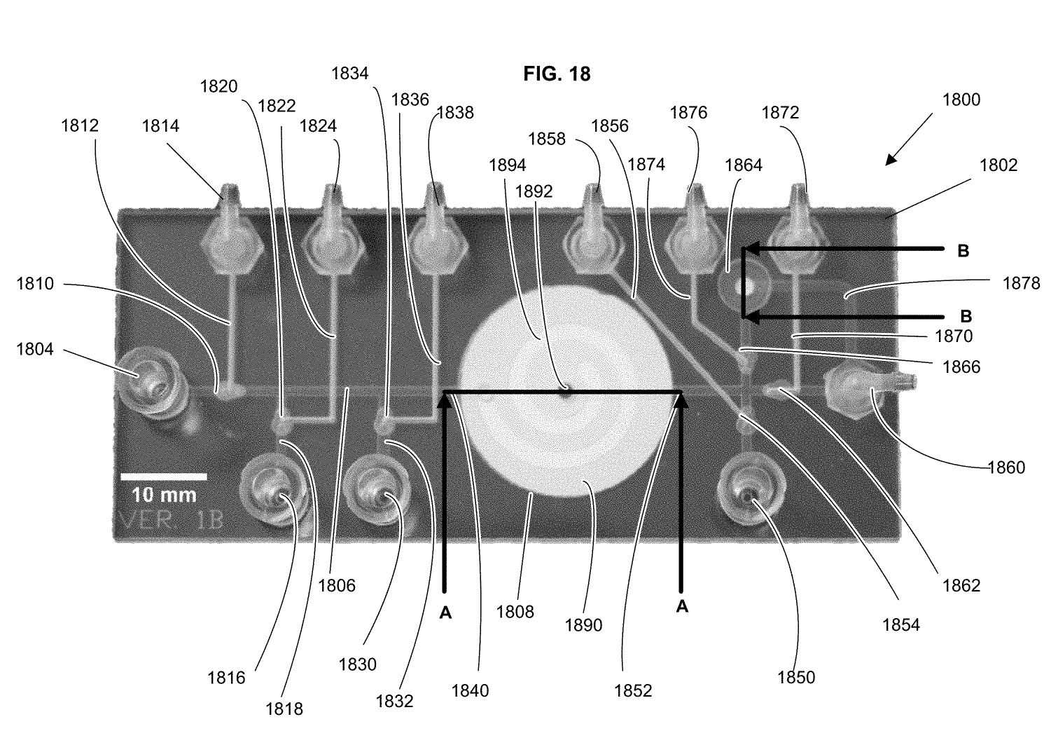

In an aspect, the various valves in microfluidic device 200 (including, but not limited to first reagent control valve 216 and detection chamber control valve 226) include pneumatically controlled valves. In this case, microfluidic device 200 also includes at least one air channel (for example as illustrated herein below in FIG. 18) for connecting at least one pneumatic pressure source to each such pneumatically controlled valve. In an aspect, air channels used to control pneumatically controlled valves have dimensions of about 1 mm wide and 100 μm high. In an aspect, microvalves are diaphragm valves. Pneumatically controlled diaphragm valves may be, for example, as described in U.S. Pat. No. 7,607,641 to Yuan or U.S. Pat. No. 6,431,212 to Hayenga et al, both of which are incorporated herein by reference. Other types of microvalves may be used, as well, and microfluidic devices as described herein are not limited to use with any specific type of microvalve.

In an aspect, microfluidic device 200 includes at least one air port 230 fluidically connected to the upstream end 210 of first filter chamber 204 and adapted for connection to a negative pressure (vacuum) source (not shown), e.g. to draw fluid into first filter chamber 204. As used herein, the “upstream end” of first filter chamber 204 refers to upstream of first filter 206, but not upstream of an inlet to the filter chamber. Further detail regarding the configuration of first filter chamber 204 is provided herein below. In an aspect, vent control valve 232 controls the flow of air through air port 230. In some aspects, air port 230 may be vented to the atmosphere to release excess pressure within first filter chamber 204. Alternatively, a positive pressure source may be attached to air port 230 to increase a pressure within first filter chamber 204 and/or drive fluid out of first filter chamber 204. The same approach for venting and/or modifying pressure can be used with the second filter chamber, though not specifically depicted or described herein.

In an aspect, microfluidic device 200 includes at least one waste port 234 located downstream of first filter chamber 204 and adapted to receive fluid waste from the downstream end 236 of the first filter chamber 204, and at least one waste control valve 238 adapted to control a flow of fluid waste from downstream end 236 of first filter chamber 204 to at least one waste port 234. For example, in an aspect the at least one waste port 234 is adapted for connection to at least one negative pressure source (not shown).

In an aspect, microfluidic device 200 includes at least one at least one waste port 234 located downstream of second filter chamber 220 and adapted to receive fluid waste from the downstream end 240 of second filter chamber 220. As depicted in FIG. 2, the waste port can be the same one used to receive waste fluid from first filter chamber 204 (i.e., waste port 234). Alternatively, a separate waste port may be used. In an aspect, such a waste port is adapted for connection to a negative pressure source for drawing waste fluid into the waste port.

In an aspect, first reagent inlet port 214 is adapted to receive the first reagent from a reagent source, which may be, for example, a reservoir of liquid reagent external to the microfluidic device. In an aspect, microfluidic device 200 includes a reservoir containing lyophilized reagent in fluid communication with the at least one reagent inlet port (e.g. reservoir 242 depicted in FIG. 2), wherein the at least one first reagent inlet port 214 is adapted to receive a fluid adapted to rehydrate the lyophilized reagent to produce the first reagent for delivery to the first filter chamber.

In an aspect, microfluidic device 200 includes at least one second reagent inlet port 250 located upstream of first filter chamber 204 and in fluid communication with upstream end 210 of first filter chamber 204, the at least one said second reagent inlet port 250 adapted to deliver to the first filter chamber a second reagent, and at least one second reagent control valve 252 adapted to control a flow of the second reagent from second reagent inlet port 250 to upstream end 210 of the first filter chamber 204.

In an aspect, microfluidic device 200 includes a reservoir (not shown, but like reservoir 242) containing lyophilized second reagent in fluid communication with second reagent inlet port 250, where second reagent inlet port 250 is adapted to receive a fluid adapted to rehydrate the lyophilized second reagent to produce the second reagent for delivery to first filter chamber 204.

In an aspect, microfluidic device 200 includes at least one third reagent inlet port 256 located upstream of first filter chamber 204 and in fluid communication with the upstream end 210 of first filter chamber 204, the at least one said third reagent inlet port 256 adapted to deliver to first filter chamber 204 a third reagent, and at least one third reagent control valve 258 adapted to control a flow of the third reagent from third reagent inlet port 256 to the upstream end 210 of first filter chamber 204. In an aspect, microfluidic device 200 includes a reservoir (not shown, but like reservoir 242) containing lyophilized third reagent in fluid communication with the at least one third reagent inlet port 256, wherein the at least one third reagent inlet port 256 is adapted to receive a fluid capable of rehydrating the lyophilized third reagent to produce the third reagent for delivery to first filter chamber 204.

In an aspect, microfluidic device 200 also includes a bypass channel 258 fluidically connecting third reagent inlet port 256 to the downstream end 236 of first filter chamber 204 and the upstream end 262 of second filter chamber 220, and a bypass valve 264 adapted to control a flow of the third reagent from the third reagent inlet port 256 to the downstream end 236 of first filter chamber 204 and upstream end 262 of second filter chamber 220.

In an alternative configuration, the third reagent inlet port is in fluid communication with the downstream end of the first filter chamber and the upstream end of the second filter chamber, so that the third reagent can be delivered from the third reagent inlet port to the second filter chamber, and the third reagent control valve is adapted to control a flow of the third reagent from the third reagent inlet port to the upstream end of the second filter chamber. This is circuit configuration is obtained by modifying the fluid circuity depicted in FIG. 2 by removing third reagent control valve and the fluid channel connecting third reagent inlet port 256 to the upstream end 210 of first filter chamber 204. Examples of such configurations can be seen, e.g. in the devices depicted in FIGS. 18 and 21.

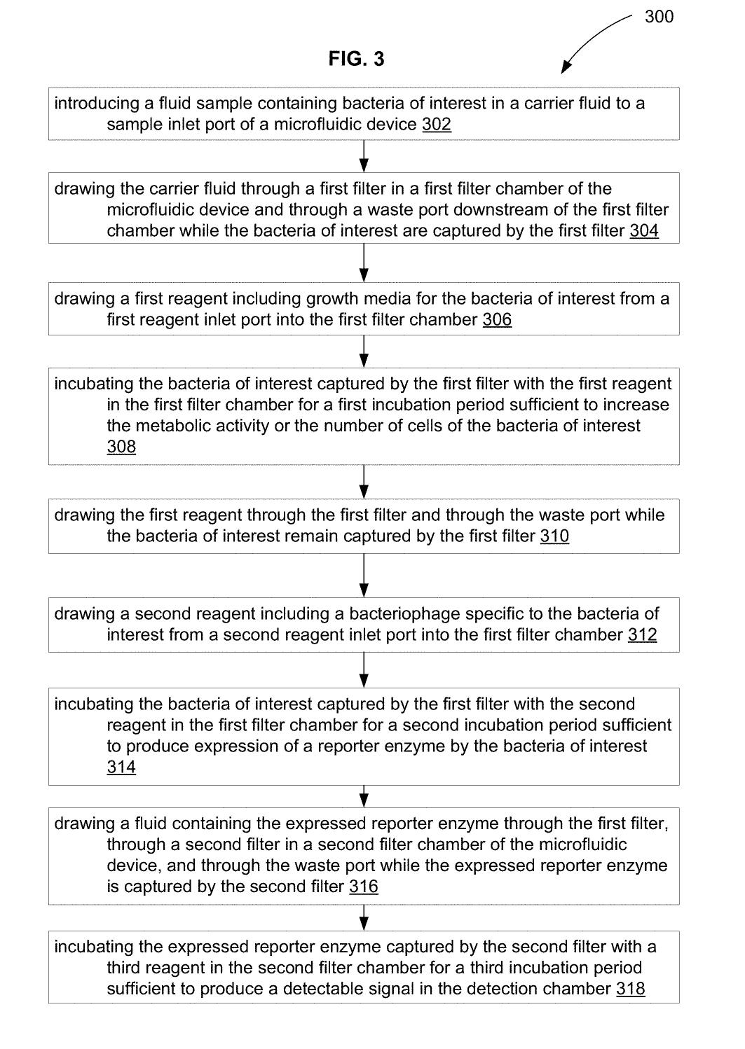

FIG. 3 is a flow diagram of a method 300 of concentrating bacteria for detection, comprising, which can be performed using a microfluidic device as depicted in FIG. 2. Method 300 includes introducing a fluid sample containing bacteria of interest in a carrier fluid to a sample inlet port of a microfluidic device, at 302; drawing the carrier fluid through a first filter in a first filter chamber of the microfluidic device and through a waste port downstream of the first filter chamber while the bacteria of interest are captured by the first filter, at 304; drawing a first reagent including growth media for the bacteria of interest from a first reagent inlet port into the first filter chamber, as indicated at 306; incubating the bacteria of interest captured by the first filter with the first reagent in the first filter chamber for a first incubation period sufficient to increase at least one of the metabolic activity or the number of cells of the bacteria of interest, at 308; drawing the first reagent through the first filter and through the waste port while the bacteria of interest remain captured by the first filter, at 310; drawing a second reagent including a bacteriophage specific to the bacteria of interest from a second reagent inlet port into the first filter chamber, at 312; incubating the bacteria of interest captured by the first filter with the second reagent in the first filter chamber for a second incubation period sufficient to produce expression of a reporter enzyme by the bacteria of interest, at 314; drawing a fluid containing the expressed reporter enzyme through the first filter, through a second filter in a second filter chamber of the microfluidic device, and through the waste port while the expressed reporter enzyme is captured by the second filter, at 316; and incubating the expressed reporter enzyme captured by the second filter with a third reagent in the second filter chamber for a third incubation period sufficient to produce a detectable signal in the detection chamber, at 318.

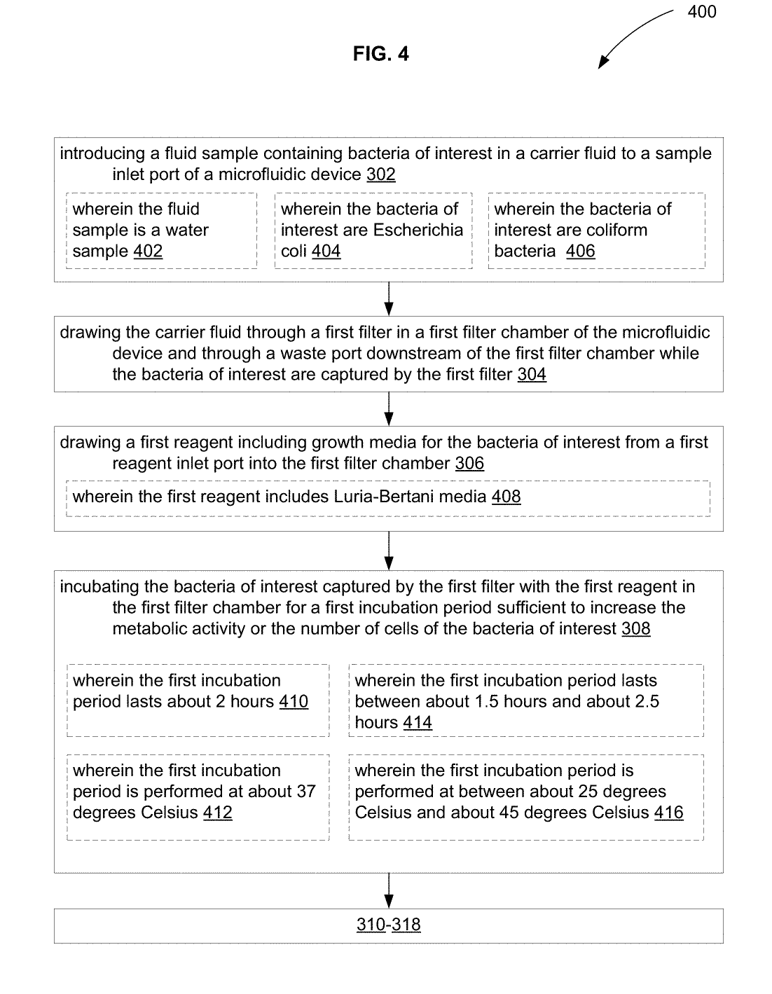

Further method aspects are shown in FIGS. 4-9. In these figures, steps 302-318 are as described in connection with FIG. 3. Optional and alternative steps are outlined with dashed lines.

FIG. 4 depicts a method 400, including further aspects relating to the bacterial sample and first incubation. In an aspect, the fluid sample is a water sample, as indicated at 402. In various aspects, bacteria of interest are Escherichia coli, as indicated at 404, or more generally, coliform bacteria, as indicated at 406. In an aspect, the first reagent includes Luria-Bertani media, as indicated at 408. Various other bacterial growth media may be used, as known to those having ordinary skill in the art. The first incubation period lasts about 2 hours at a temperature of about 37 degrees Celsius, for example, as indicated at 410, and 412, respectively. More generally, the first incubation period may last between about 1.5 hours and about 2.5 hours, as indicated at 414, and be between about 25 degrees Celsius and about 45 degrees Celsius, as indicated at 416.

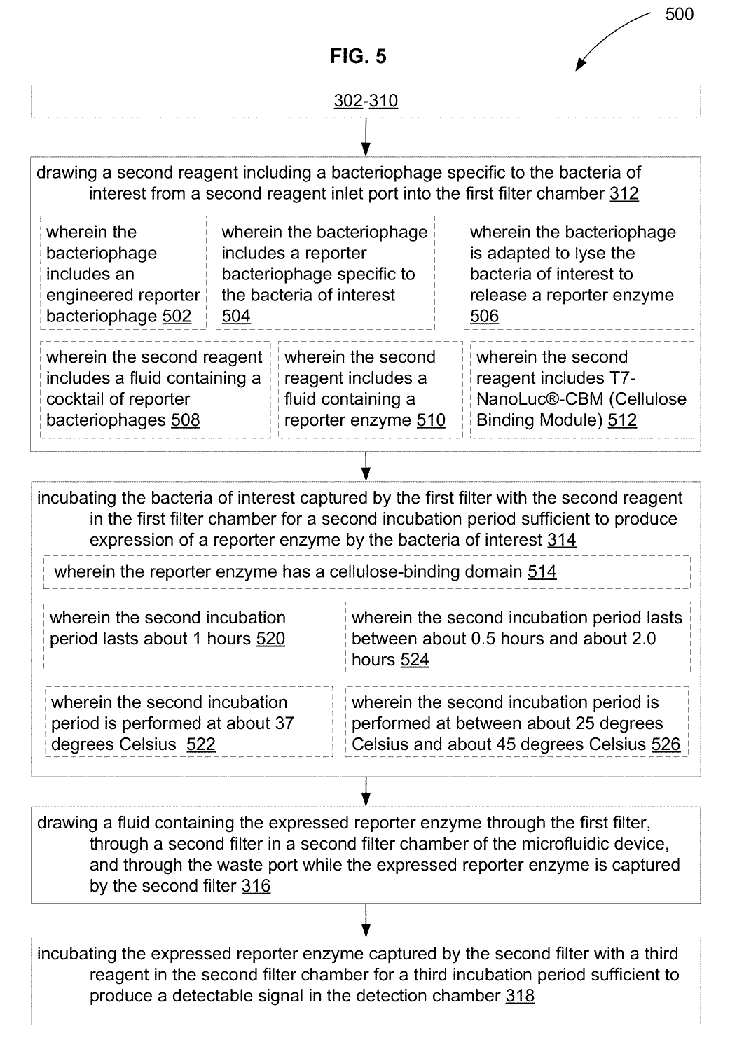

FIG. 5 depicts a method 500, including further aspects relating to the second reagent and incubation period. In various aspects, the bacteriophage includes an engineered reporter bacteriophage, as indicated at 502 and/or a reporter bacteriophage specific to the bacteria of interest, as indicated at 504. In an aspect, the bacteriophage is adapted to lyse the bacteria of interest to release a reporter enzyme, as indicated at 506. In another aspect, the second reagent includes a fluid containing a cocktail of reporter bacteriophages, as indicated at 508. In some aspects, the second reagent includes a fluid containing a reporter enzyme, as indicated at 510. As an example, the second reagent includes T7-NanoLuc®-CBM (Cellulose Binding Module), as indicated at 512.

Method 500 includes incubating the bacteria of interest captured by the first filter with the second reagent in the first filter chamber for a second incubation period sufficient to produce expression of a reporter enzyme by the bacteria of interest 314, as discussed herein above. In an aspect, the reporter enzyme has a cellulose-binding domain, as indicated at 514. In an aspect, the second incubation period lasts about 1 hours, as indicated at 520, and is performed at about 37 degrees Celsius, as indicated at 522. More generally, the second incubation period may last between about 0.5 and about 2.0 hours, as indicated at 524, and may be performed at between about 25 degrees Celsius and about 45 degrees Celsius, as indicated at 526.

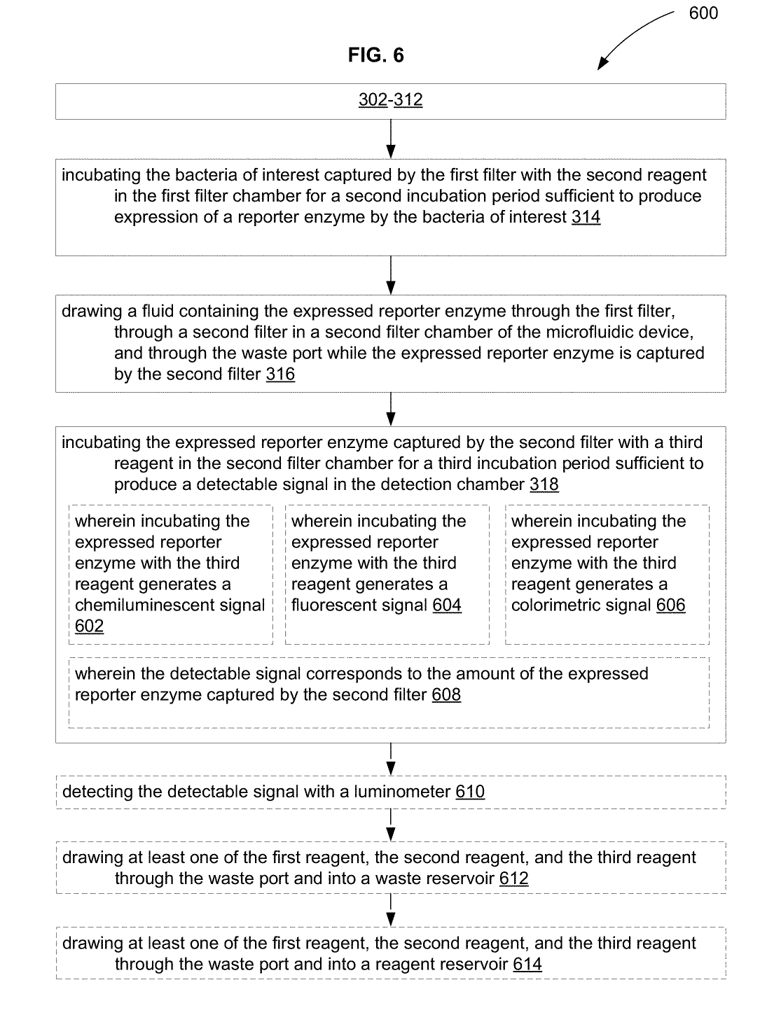

FIG. 6 depicts a method 600, including further aspects relating the third incubation. In various aspect, incubating the expressed reporter enzyme with the third reagent generates a chemiluminescent signal, as indicated at 602, a fluorescent signal, as indicated at 604, or a colorimetric signal, as indicated at 606. In an aspect, the detectable signal corresponds to the amount of the expressed reporter enzyme captured by the second filter, as indicated at 608. The detectable signal can be detected with a luminometer, as indicated at 610, or with other equipment capable of detecting an optical signal. In an aspect, the detectable signal may be in a non-visible portion of the electromagnetic spectrum, and equipment suitable for detecting other electromagnetic signals may be used.

FIG. 6 also includes steps relating to handling of excess fluids after they have passed through the waste port. In some aspects, method 600 includes drawing at least one of the first reagent, the second reagent, and the third reagent through the waste port and into a waste reservoir, as indicated at 612. In other aspects, method 600 includes drawing at least one of the first reagent, the second reagent, and the third reagent through the waste port and into a reagent reservoir, as indicated at 614. As discussed herein above, waste reagents can be collected in a reagent reservoir and reused. In particular, in an aspect, a water sample which has previously passed through the first filter can be used to rehydrate lyophilized reagent to produce a second reagent for introduction into the first filter. Alternatively, rather than recycling the solvent (water) component of the reagent, the solute component of the reagent may be collected, either for reuse or to prevent release into the environment in the case that it includes a hazardous material.

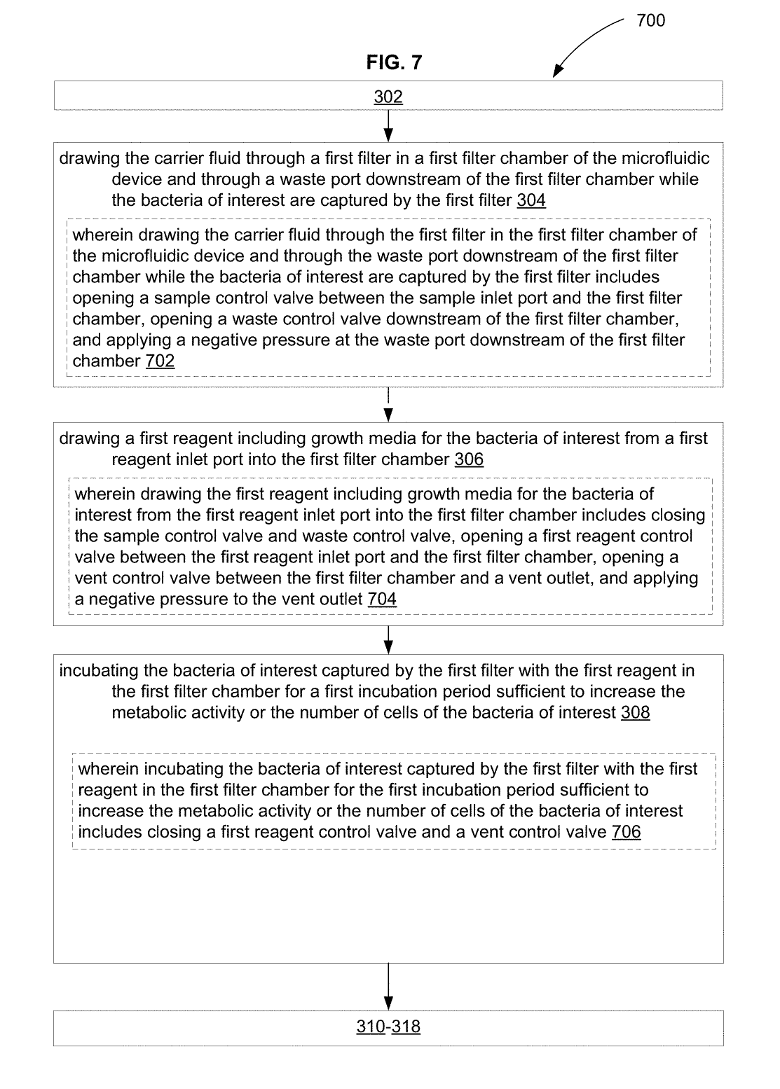

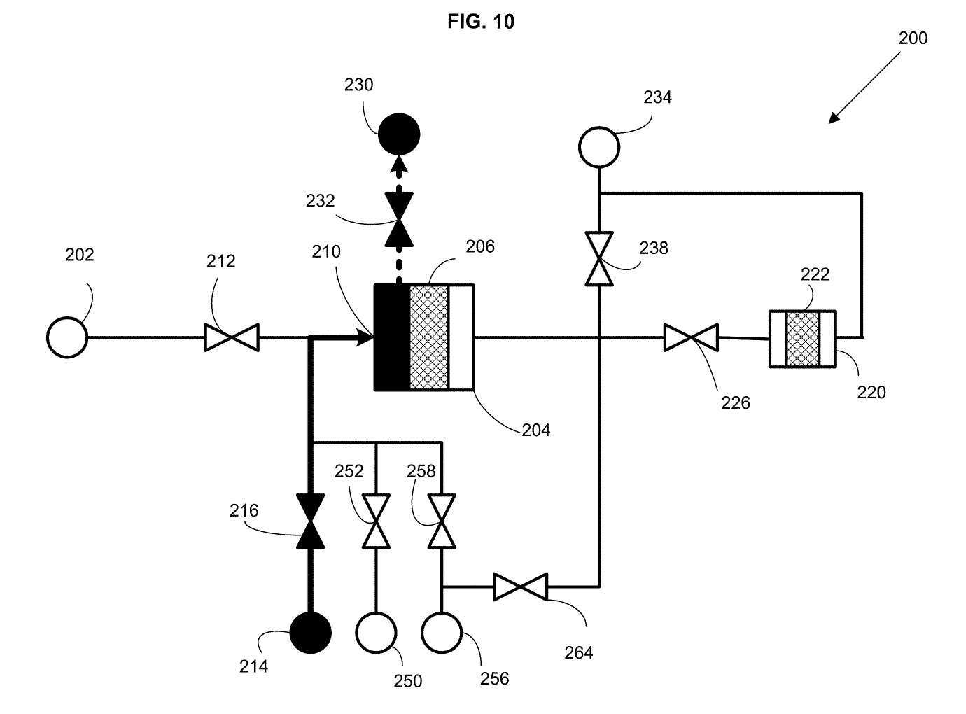

FIG. 7 depicts a method 700 providing further detail of aspects of fluid handling in the microfluidic device. Performance of method 700 with microfluidic device 200 is illustrated in FIG. 2. In FIG. 2 and FIGS. 10-17, which are discussed herein below, fluid flow is indicated by heavy black lines, air flow is indicated by heavy dashed lines, open valves are indicated in black, and closed valves are indicated in white. Components identified by reference numbers in FIGS. 10-17 are as described above in connection with FIG. 2. As indicated at 702 in FIG. 7, and illustrated in FIG. 2, drawing the carrier fluid from 202 through the first filter 206 in the first filter chamber 204 of the microfluidic device and through the waste port 234 downstream of the first filter chamber 204 while the bacteria of interest are captured by the first filter 206 includes opening a sample control valve 212 between the sample inlet port 202 and the first filter chamber 204, opening a waste control valve 238 downstream of the first filter chamber 204, and applying a negative pressure at the waste port 234 downstream of the filter chamber, as indicated at 702 in FIG. 7.

In addition, as shown in FIG. 7 at 704, and illustrated in FIG. 10, in an aspect, drawing the first reagent including growth media for the bacteria of interest from the first reagent inlet port 214 into the first filter chamber 204 includes closing the sample control valve 212 and waste control valve 238, opening a first reagent control valve 216 between the first reagent inlet port 214 and the first filter chamber 204, opening a vent control valve 232 between the filter chamber 204 and a vent outlet (air port 230), and applying a negative pressure to the vent outlet (air port 230).

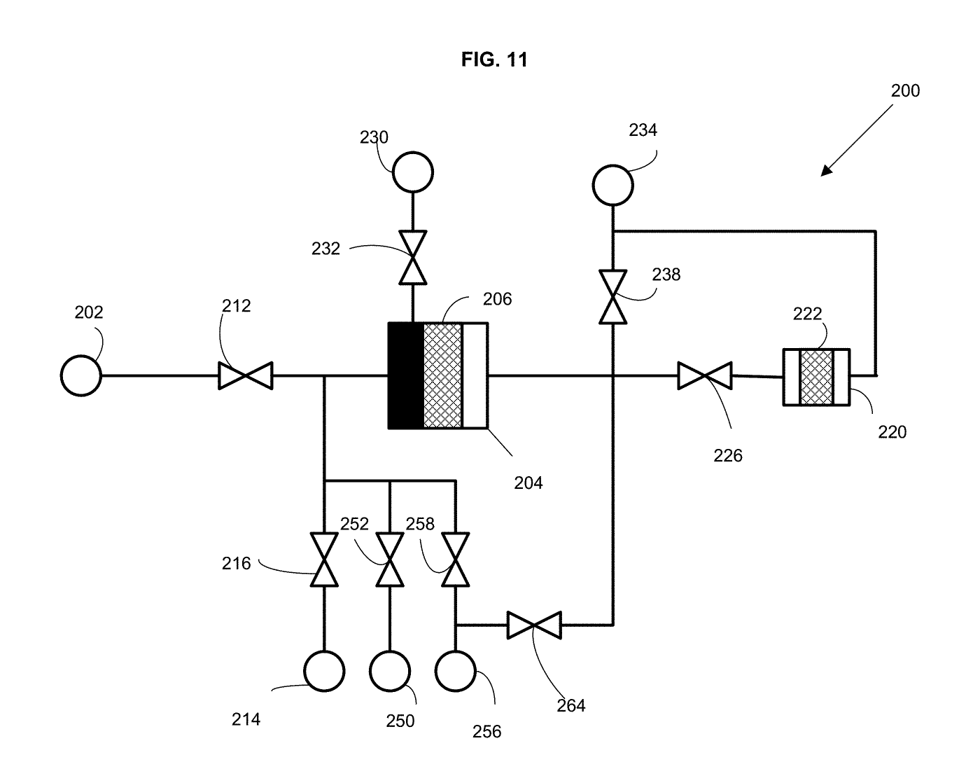

In a further aspect, as shown in FIG. 7 at 706, and illustrated in FIG. 11, incubating the bacteria of interest captured by the first filter 206 with the first reagent in the first filter chamber 204 for the first incubation period sufficient to increase at least one of the metabolic activity or the number of cells of the bacteria of interest includes closing a first reagent control valve 216 and a vent control valve 232.

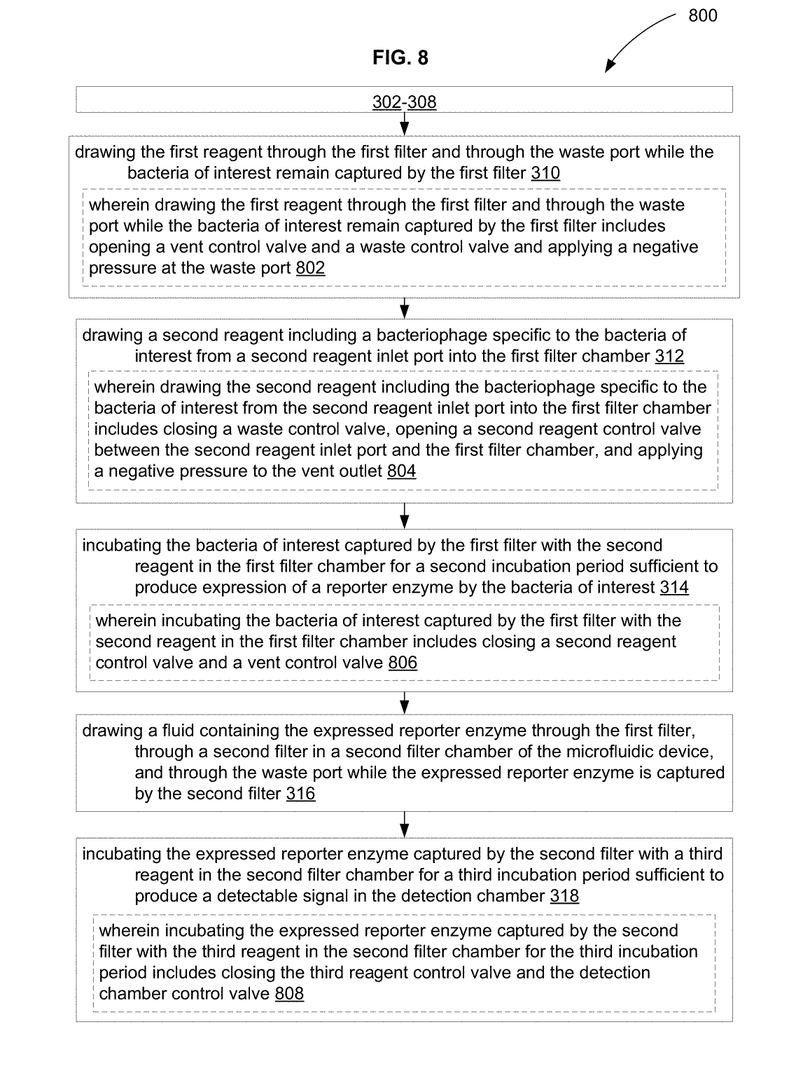

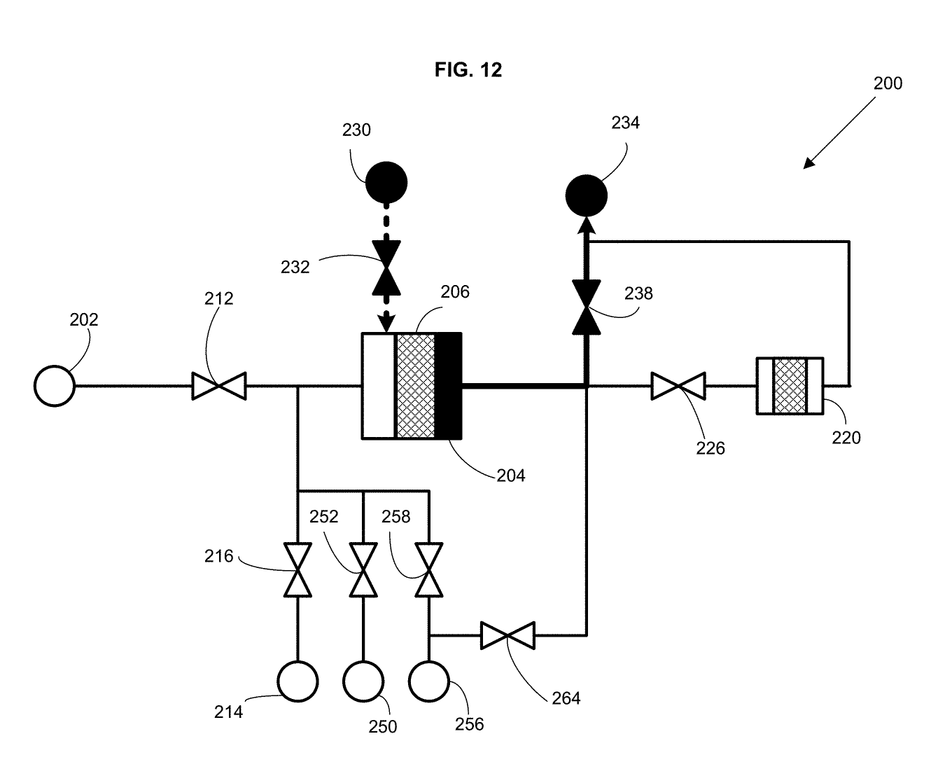

FIG. 8 is a flow diagram of a method 800 relating to further fluid handling aspects. In a further aspect, as shown in FIG. 8 at 802 and illustrated in FIG. 12, drawing the first reagent through the first filter 206 and through the waste port 234 while the bacteria of interest remain captured by the first filter 206 includes opening a vent control valve 232 and a waste control valve 238 and applying a negative pressure at the waste port 234.

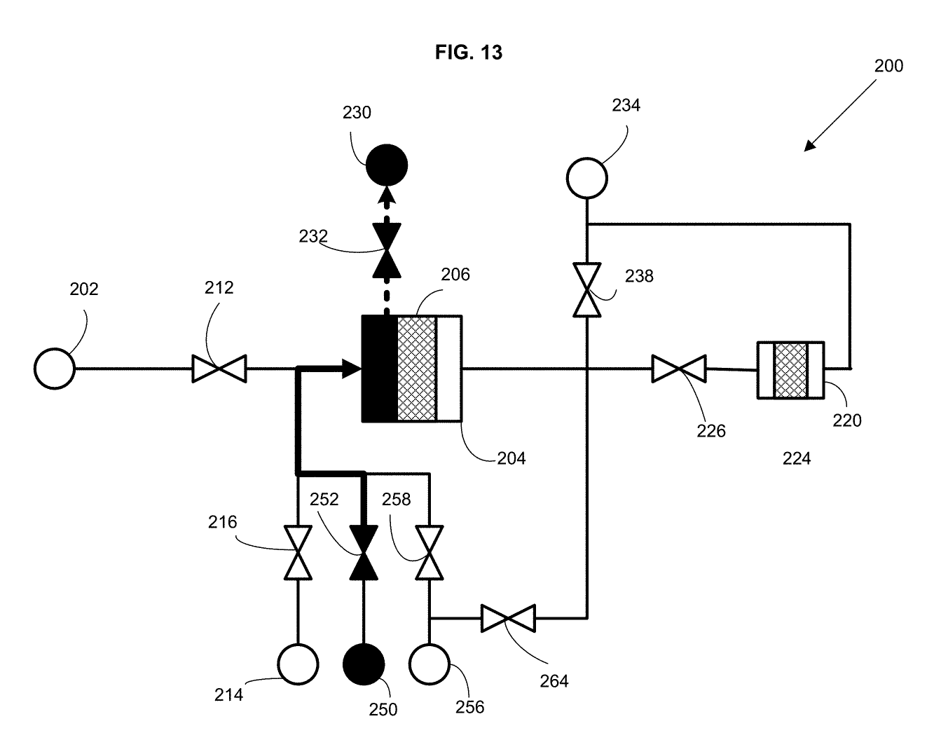

In a further aspect, as shown in FIG. 8 at 804 and illustrated in FIG. 13, drawing the second reagent including the bacteriophage specific to the bacteria of interest from the second reagent inlet port 250 into the first filter chamber 204 includes closing waste control valve 238, opening a second reagent control valve 252 between the second reagent inlet port 250 and the first filter chamber 204, and applying a negative pressure to the vent outlet (air port 230).

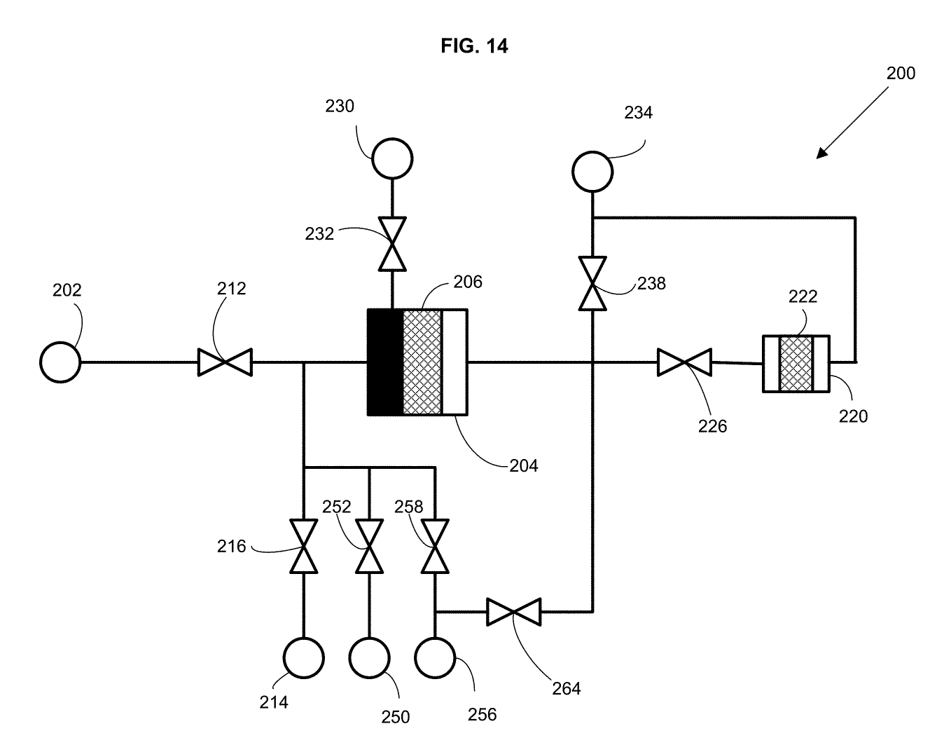

In a further aspect, as shown in FIG. 8 at 806 and illustrated in FIG. 14, incubating the bacteria of interest captured by the first filter 206 with the second reagent in the first filter chamber 204 includes closing a second reagent control valve 252 and a vent control valve 232.

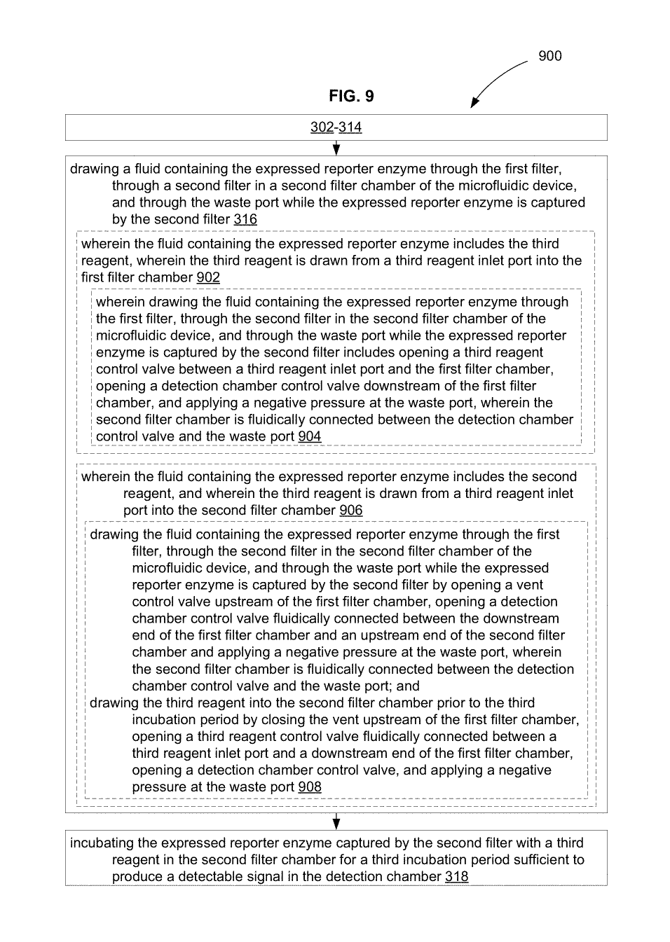

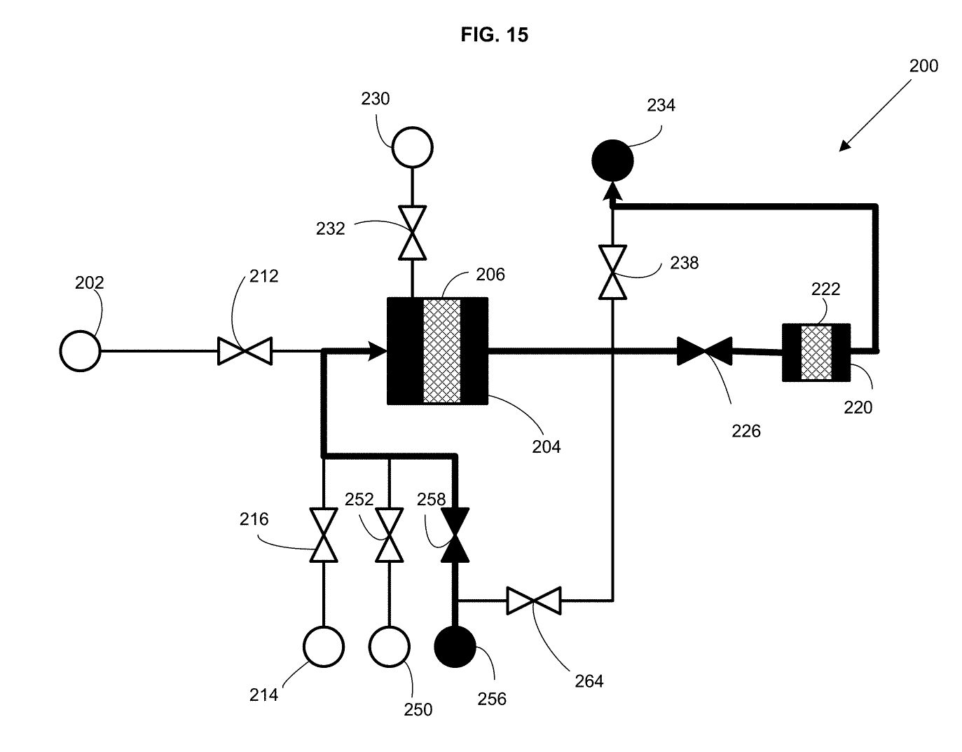

FIG. 9 is a flow diagram showing further aspects of a method 900 of concentrating bacteria for detection. In an aspect, as shown in FIG. 9 at 902, and illustrated in FIG. 15, the fluid containing the expressed reporter enzyme includes the third reagent, wherein the third reagent is drawn from a third reagent inlet port 256 into the first filter chamber 204, as indicated at 902. For example, in an aspect, drawing the fluid containing the expressed reporter enzyme through the first filter 206, through the second filter 222 in the second filter chamber 220 of the microfluidic device, and through the waste port 234 while the expressed reporter enzyme is captured by the second filter 222 includes opening a third reagent control valve 258 between a third reagent inlet port 256 and the first filter chamber 204, opening a detection chamber control valve 226 downstream of the first filter chamber 204, and applying a negative pressure at the waste port 234, wherein the second filter chamber 220 is fluidically connected between the detection chamber control valve 226 and the waste port 234, as indicated at 904 in FIG. 9.

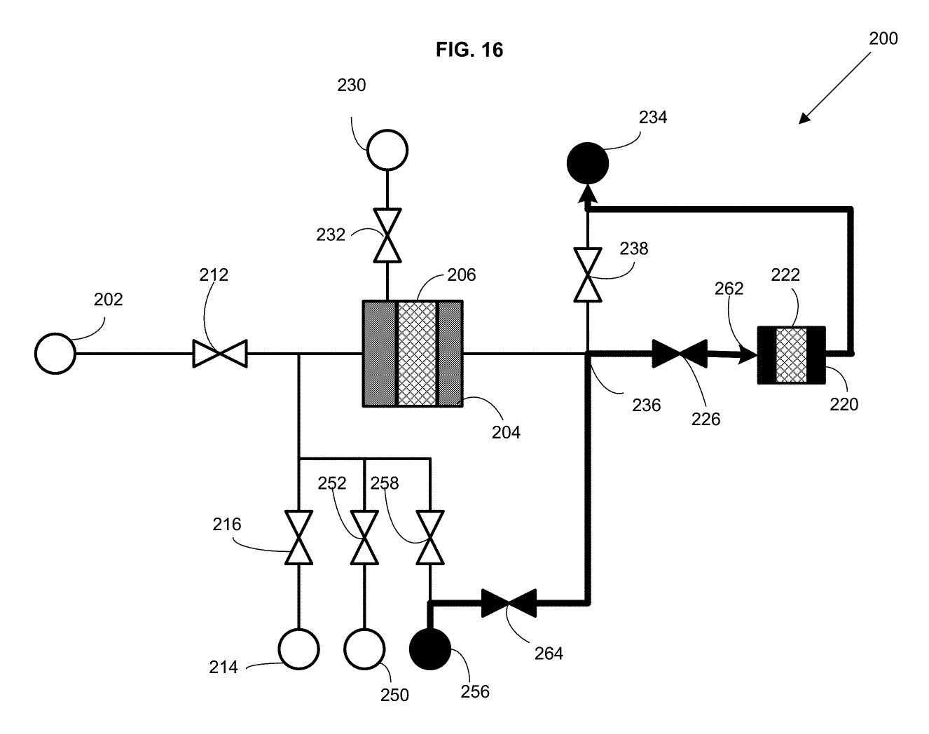

Alternatively, as shown in FIG. 9 at 906, the fluid containing the expressed reporter enzyme includes the second reagent (here, the fluid remaining in the first filter chamber following the second incubation), and wherein the third reagent is drawn from a third reagent inlet port 256 into the second filter chamber 220. For example, as shown in FIG. 9 at 908, in an aspect this can be accomplished by drawing the fluid containing the expressed reporter enzyme through the first filter, through the second filter in the second filter chamber of the microfluidic device, and through the waste port while the expressed reporter enzyme is captured by the second filter. This could be done by opening vent control valve 232 upstream of first filter chamber 204, opening detection chamber control valve 226 fluidically connected between the downstream end 236 of the first filter chamber 204 and an upstream end 262 of the second filter chamber 220, and applying a negative pressure at waste port 234. As shown in FIG. 9 at 908, and illustrated in FIG. 16, the third reagent is drawn from a third reagent inlet port 256 into the second filter chamber 220 prior to the third incubation period by closing the vent control valve 232, opening a third reagent control valve (here, bypass valve 264) fluidically connected between a third reagent inlet port 256 and a downstream end 236 of the first filter chamber 204, opening a detection chamber control valve 226, and applying a negative pressure at the waste port 234, wherein the second filter chamber 220 is fluidically connected between the detection chamber control valve 226 and the waste port 234.

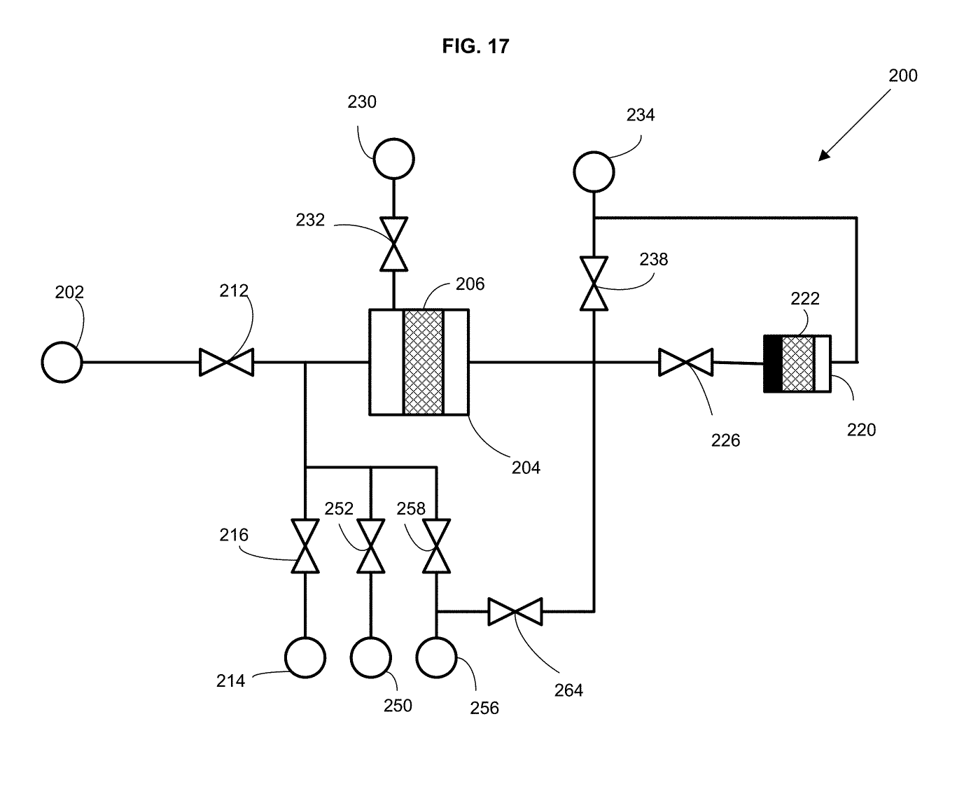

In a further aspect, as shown in FIG. 8 at 808 and illustrated in FIG. 17, incubating the expressed reporter enzyme captured by the second filter 222 with the third reagent in the second filter chamber 220 for the third incubation period includes closing the third reagent control valve 258 and the detection chamber control valve 226. Following the incubation period, a detectable signal is detected from second filter chamber 220.

FIG. 18 is a photograph of an example of a microfluidic device 1800 containing fluid circuitry for performing a method as described in connection with FIGS. 2 and FIG. 4-9. In an aspect, microfluidic device 1800 is used for detecting E. coli in a water sample. FIG. 18 is top view of microfluidic device 1800. In an aspect, in use, microfluidic device 1800 is placed on a horizontal surface, with the surface visible in FIG. 18 facing upward. Alternatively, in some aspects microfluidic devices as described herein may be oriented vertically, e.g. to reduce footprint and/or to process more samples in parallel. Microfluidic device 1800 is formed from a laminated polymeric substrate 1802. In the example of FIG. 18, microfluidic device 1800 is formed of polycarbonate sandwiched between layers of acrylic. Layers are adhered together by a pressure sensitive adhesive. Layers are aligned and adhered together. Construction of microfluidic device 1800 is described in greater detail herein below.

Sample inlet port 1804 includes an attached Luer lock that permits a syringe filter or cup containing sample fluid to be interfaced with microfluidic device 1800. Sample fluid travels from sample inlet port 1804 through fluid channel 1806 to first filter chamber 1808. Flow of sample fluid is controlled by sample control valve 1810, which is a pneumatically controlled valve. Air channel 1812 connects to air port 1814 which is configured for connection with a pneumatic pressure source for controlling sample control valve 1810. In microfluidic device 1800, air port 1814 includes a hose barb that can be connected to a line leading to a pneumatic pressure source. Alternatively, air ports can be configured for connection to a pneumatic pressure source by having a smooth surface around the air port, to which an o-ring or other seal-forming element can be pressed or clamped to form a sealed connection.

First reagent inlet port 1816 includes a Luer lock. First reagent inlet port 1816 is connected to fluid channel 1806 by channel 1818. First reagent control valve 1820 is controlled via air channel 1822 connected to air port 1824. Second reagent inlet port 1830 also includes a Luer lock. Second reagent inlet port 1830 is connected to fluid channel 1806 by channel 1832. Second reagent control valve 1834 is controlled via air channel 1836 connected to air port 1838. First reagent inlet port 1816 and second reagent inlet port 1830 are fluidically connected to the upstream end 1840 of first filter chamber 1808. Third reagent inlet port 1850 is fluidically connected to the downstream end 1852 of first filter chamber 1808. This is allows for delivery of third reagent in the manner depicted in FIG. 16. Third reagent control valve 1854 is controlled via air channel 1856 leading to air port 1858. From downstream end 1852 of first filter chamber 1808, fluid can be delivered to waste port 1860 under control of waste control valve 1862, or second filter chamber 1864 under control of detection chamber control valve 1866. Waste control valve 1862 is controlled via air channel 1870 to air port 1872, and detection chamber control valve 1866 is controlled via air channel 1874 to air port 1876. Channel 1878 provides for waste fluid and/or air to be drawn from the downstream end of second filter chamber 1864 to waste port 1860.

Air ports 1824, 1838, 1858, 1872, and 1876 include hose barbs for connecting to a pneumatic pressure source for controlling valve operation. Waste port 1860 also includes a hose barb, for connection to a negative pressure source. As noted above, a fluid reservoir (not shown; external to microfluidic device 1800) may be associated with waste port 1860, to collect fluid exiting waste port 1860. Sample inlet port 1804 and reagent inlet ports 1816, 1830 and 1850 include Luer locks for interfacing with fluid sources.

First filter chamber 1808 has flattened cylinder shape to accommodate filter 1890, which is disk shaped with a central hole 1892. Filter 1890 is formed from polyvinyldifluoride, with a thickness of 110-150 μm and pore size of about 0.45 μm (available from Sterlitech Corporation, Kent, Wash.). Filter 1890 captures E. coli from the fluid sample. A spiral channel 1894 in the upper surface of first filter chamber 1808 distributes fluid rapidly over the top surface of filter 1890, within the spiral channel 1894, before it spreads laterally and downward through filter 1890. The function of the first filter is to filter the bacteria from the environmental sample. In an aspect, it is desired to process at least 100 mL within a relatively short period of time (e.g., few minutes).

Filtration time is influenced by membrane pore size (here, 0.45 μm or smaller), channel aspect ratio, channel length-membrane size, and effective filtering area, which is depends upon spiral channel geometry. At the same time, it is desired to reducing adverse protein interactions (enzyme binding) and minimizing device footprint, to enhance portability of the device.

In an aspect, the first filter has an area of 315 mm². The area of the spiral channel above the first filter is 200 mm². The channel is 200 μm high, giving a channel volume of 40 μl. Hypothetically, the channel area above the filter can accommodate, in a single layer, about 0.2 mm³ or 0.2 mg of bacteria (assuming bacteria are E. coli, each having dimensions of 0.5 μm×2 μm and mass of 1 pg).

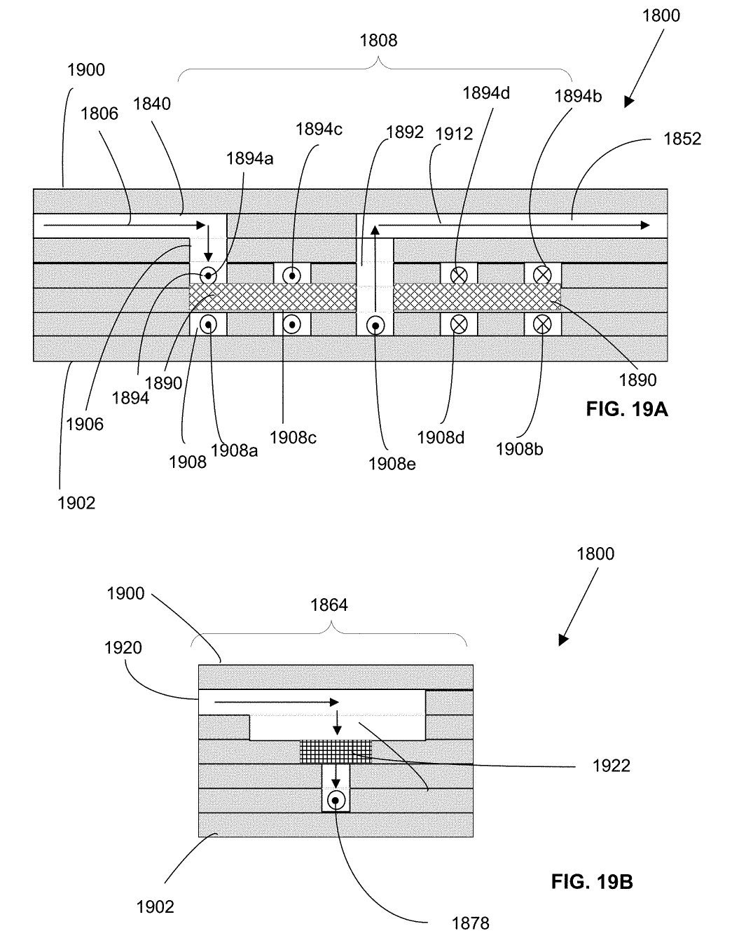

The construction of first filter chamber 1808 can be understood with reference to FIG. 19A, which is a cross-sectional side view of first filter chamber 1808, taken at section line A-A in FIG. 18. The top surface of the microfluidic device 1800 is indicated at 1900, and the bottom surface is indicated at 1902. Fluid enters at the top of first filter chamber 1808 from fluid channel 1806 at upstream end 1840 from fluid channel 1806, and exits at downstream end 1852. The direction of fluid flow is indicated by arrows in FIG. 19A. As can be seen, fluid channel 1806 is formed in a second layer of microfluidic device 1800. Fluid travels through via 1906 from fluid channel 1806 to spiral channel 1894. In FIG. 19A, fluid flow out of the plane of the page is indicated by a circle containing a dot, and fluid flow into the plane of the page is indicated by a circle containing an X. Fluid flows in spiral channel 1894 sequentially through segments 1894a, 1894b, 1894c and 1894d. At the same time, fluid penetrates through filter 1890 to a corresponding channel 1908 on the lower surface of filter chamber 1808, where it flows through segments 1908a, 1908b, 1908c, 1908d, and 1908e. Channel 1908 collects fluid that has passed through filter 1890. Fluid then passes through central hole 1892 to channel 1912 that exits downstream of the filter at the center of first filter chamber 1808. As can be seen in FIG. 19A, although channel 1912 exits first filter chamber 1808 in a layer above filter 1890, fluid enters channel 1912 only after it has passed through filter 1890.

FIG. 19B is a cross-sectional side view of second filter chamber 1864, taken at section line B-B in FIG. 18. The top surface of the microfluidic device 1800 is indicated at 1900, and the bottom surface is indicated at 1902. Fluid enters at the top of second filter chamber 1864 from at inlet 1920, which is fluidically connected to the downstream end 1852 of first filter chamber 1808, as shown in FIG. 18. It passes through second filter 1922 and exits via channel 1878, which as discussed herein above leads to waste port 1860, as shown in FIG. 18. Second filter 1922 is formed from nitrocellulose having a pore size of about 0.2 μm and thickness of between about 101.6 and about 190.5 μm (manufactured by Pall Industries, Port Washington, N.Y.). Second filter 1922 binds the cellulose binding module tag on the enzyme. Second filter 1922 can have different pore sizes providing it captures the reporter enzyme, e.g. by binding the cellulose binding module tag. The material forming the structure of microfluidic device 1800 is substantially transparent, hence a detectable signal produced by material in second filter chamber 1864 and/or captured by second filter 1922 can be detected through top surface 1900. In embodiments in which the main structure of the microfluidic device is formed from a material that does not transmit the detectable signal, at least one surface of the second filter chamber can be formed from a material transparent to the detectable signal, to permit detection of the detectable signal from the exterior of the microfluidic device.

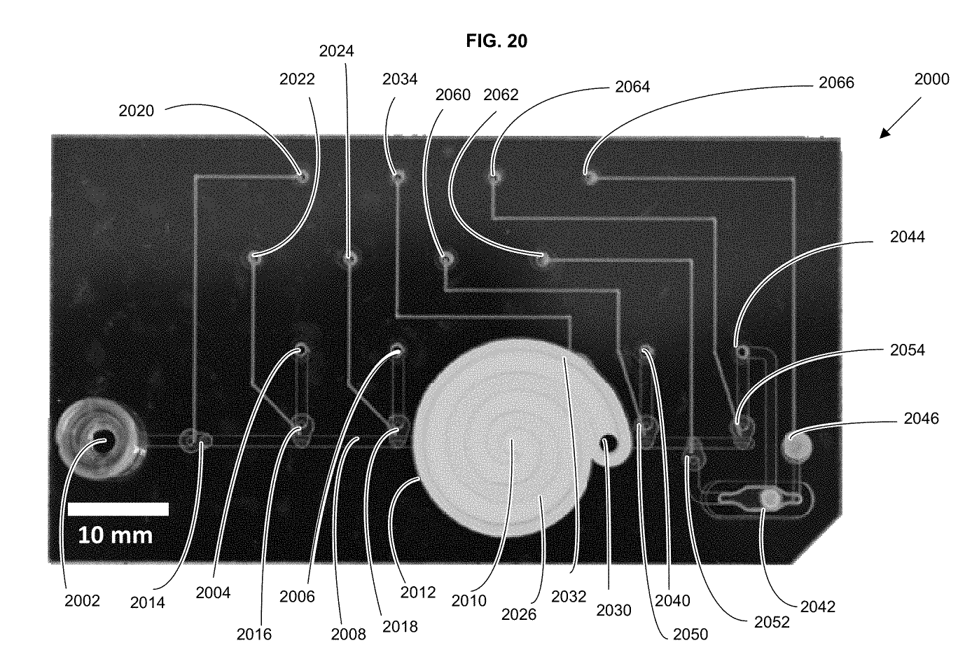

FIG. 20 depicts an alternative layout for a microfluidic device 2000 for performing fluid handling steps substantially similar to those performed by the microfluidic device of FIG. 18. Microfluidic device 2000 includes sample inlet port 2002, first reagent inlet port 2004, and second reagent inlet port 2006, connected to channel 2008 leading to inlet 2010 of first filter chamber 2012. Sample control valve 2014, first reagent control valve 2016 and second reagent control valve 2018 are controlled via air ports 2020, 2022, and 2024, respectively. Spiral channel 2026 runs from inlet 2010 to outlet 2030. As described in connection with FIG. 18, spiral channel 2026 is on the upstream side of the first filter chamber 2012 (i.e., on a first side of the filter, which is not depicted in FIG. 20, but as described in connection with FIG. 18). A corresponding spiral channel (not shown) is on the downstream side of the first filter chamber (i.e., on a second side of the filter). Outlet 2030 is located on the downstream side of the first filter chamber 2012, and receives fluid that has passed through the first filter and entered the spiral channel on the downstream side of the filter. Vent 2032 is located on the first (upstream) side of the first filter chamber, at a distal end of spiral channel 2026, such that a vacuum applied to vent 2032 (via air port 2034) causes fluid to flow into spiral channel 2026, as described in connection with step 806 of FIG. 8. In addition, air port 2034 can be opened to permit fluid to be drawn through the first filter and into the second filter chamber, e.g. as described in connection with step 908 of FIG. 9. Microfluidic device 2000 also includes third reagent inlet port 2040, second filter chamber 2042, outlet port 2044, and vent 2046. Fluid flow downstream of first filter chamber 2012 is controlled by third reagent control valve 2050, detection chamber control valve 2052, and waste control valve 2054, controlled via air ports 2060, 2062, and 2064, respectively. Air port 2066 is connected to vent 2046. It will be appreciated that the microfluidic devices depicted in FIGS. 18 and 20 provide two different layouts for performing substantially the same fluid handling functions. The devices differ in the arrangements of air ports and fluid inlets and outlets on the device, and differ slightly in venting arrangement. For example, other configurations may be used to optimize particular aspects of device performance or reduce device footprint.

Microfluidic devices as described herein can be attached to fluid sources supplying sample and reagent fluids, to pneumatic control lines for controlling operation of pneumatic valves, and one or more negative pressure source with associated waste or reagent reservoir for collecting fluid that has passed through the device. In an aspect, a microfluidic device includes attached hose barbs and/or Luer locks for connecting to air or fluid sources, as shown in FIG. 18. In other aspects, air or fluid sources include o-rings or other seal-forming elements that are pressed or clamped against the microfluidic device to form a sealed connection with respective air or fluid inlet openings in the device. Air or fluid sources may be connected individually to a microfluidic device, or multiple air and/or fluid sources may be connected to a microfluidic device via a manifold device that provides connection to multiple air or fluid inlet openings at the same time. Fluid waste or air vent lines may be connected to a microfluidic device in the same manner.

Pneumatic microvalves can be controlled, for example, by an ADEPT (ALine Development Platform) 12 Channel Pneumatic Controller from ALine, Inc., Rancho Dominguez, Calif., USA). The ADEPT is a programmable microfluidic controller that can operate up to 16 independent pneumatic valves under software control with programming from a computer interface, or, alternatively, by manual switches.

Incubation steps as described herein may be performed by placing the microfluidic device into an incubator. Alternatively, in an aspect, the microfluidic device may include one or more onboard heating element (e.g. a resistive element). In another aspect, the microfluidic device may be locally heated by application of energy via a laser, focused RF or ultrasonic energy, or the like.

In an aspect, multiple microfluidic devices can be processed in parallel by using a custom-built device that is adapted to interface with multiple microfluidic devices at the same time. Such a device could include, for example, positive and negative pressure sources for controlling valves and driving the flow of fluid through the device, reagent sources, and reservoirs for capturing (and optionally recycling) waste fluid. In an aspect, a reagent source could include a reservoir or liquid reagent.

As noted above, microfluidic devices as described herein can be formed from a laminated polymeric substrate. For example, in some aspects, microfluidic devices are formed from layers of polycarbonate sandwiched between layers of acrylic. Materials for use in microfluidic devices as described herein may be selected for various properties, including biocompatibility, optical clarity (for detection area) and low protein binding. In some aspects, channels and chambers are formed by laser etching; alternatively, channels and chambers can be die cut or formed by other manufacturing methods. In an aspect, layers are aligned and adhered together with a pressure sensitive adhesive (such as silicone plus tackifiers). Alternatively, other adhesive materials, such as thermally sensitive adhesives can be used. Microfluidic devices as described herein can be formed with different numbers and types of layers.

Microfluidic devices as described herein can be manufactured by various processes, for example as described in Levine, Leanna, M. “Developing Diagnostic Products Using Polymer Laminate Technology,” Aline, Inc., Redondo Beach, Calif.; and Fiorini, Gina S., Chiu, Daniel T., 2005, “Disposable microfluidic devices: fabrication, function, and application,” BioTechniques 38: 429-446, March 2005, each of which is incorporated herein by reference. In an aspect, a microfluidic device can be manufactured from cast plastic material (e.g. polydimethylsiloxane (PDMS)), e.g. as described in Friend, James and Yeo, Leslie (2010) “Fabrication of microfluidic devices using polydimethylsiloxane,” BIOMICROFLUIDICS 4, 026502, doi: 10.1063/1.3259624, which is incorporated herein by reference. For example, a device can be manufactured from laminated polymeric sheet materials by a reel-to-reel process of the type described, for example, in U.S. Published Patent Application No. 2009/0173428 to Klingbeil et al. and U.S. Pat. No. 6,375,871 to Bentsen et al., both of which are incorporated herein by reference. Devices can be made through injection molding processes, as well.

Detection of bacteria in contaminated fluid samples can be performed with different combinations of reagents. In the examples described herein, an engineered phage causes bacteria to produce an enzyme that produces luminescence when it interacts with substrate. In an aspect, the phage can be engineered to cause production of a NanoLuc® Reporter enzyme that includes a cellulose binding module tag that causes it to bind to the nitrocellulose material of the second filter. The NanoLuc® Reporter enzyme is used in combination with Nano-Glo® Luciferase Assay Reagent (the third reagent) (both obtained from Promega Corporation, Madison, Wis.) to produce a detectable signal at λ=460 nm. The luminescence can be detected with a luminometer. It will be appreciated that microfluidic devices as described herein can be configured (through appropriate selection of filter materials) to work in combination with bacteria and assay reagents other than those described specifically herein.

In the example provided herein, bacteria are lysed by the engineered phage used to induce production of the reporter enzyme. Alternatively, the microfluidic device could be modified to produce lysis of the bacteria through some other mechanism. For example, means for lysing the bacteria can include, but are not limited to, reagents such as enzymes, changing device temperature, sonication, or pressure. In an aspect, the microfluidic device includes lysing means for lysing the bacteria of interest to release the reactive material. For example, in various aspects, a lysing means includes heating means, acoustic means (e.g., a sonicator), a pressure source, a reagent source, or an enzyme source. In an aspect, the microfluidic device is configured to cooperate with an external lysing means, such as an external heat source or external acoustic source for providing sonication.

Microfluidic devices described herein utilize microfluidic means such as various combinations of microchannels, microvalves, filters, fluid or air ports, associated fluid sources, reagent reservoirs (containing liquid or lyophilized reagent materials), and positive and negative pressure sources, to perform a variety of functions, including, but not limited to, capturing bacteria of interest from the fluid sample, introducing bacterial growth media, introducing phage specific to the bacteria of interest, flushing reactive material (e.g., an enzyme) released from the bacteria of interest responsive to introduction of the phage, capturing the reactive material flushed from the first filter chamber, and performing readout of the detectable signal, It will be appreciated that various different microfluidic circuit configurations can provide equivalent functionality, and the invention is not limited to the specific fluid circuitry configurations depicted herein.

Aspects of the subject matter described herein are set out in the following numbered clauses:

Clause 1. A microfluidic device comprising:

a sample inlet port adapted to receive a fluid sample containing bacteria of interest;

a first filter chamber located downstream from the sample inlet port, the first filter chamber containing a first filter having a first area and formed from a first porous material having a pore size adapted to capture the bacteria of interest;

a sample inlet channel connecting the sample inlet port to an upstream end of the first filter chamber;

a sample control valve in the sample inlet channel, the sample control valve adapted to control a flow of the sample fluid from the sample inlet port to the upstream end of the first filter chamber;

at least one first reagent inlet port located upstream of the first filter chamber and in fluid communication with the upstream end of the first filter chamber, the at least one first reagent inlet port adapted to deliver to the first filter chamber a first reagent containing a bacteriophage specific to the bacteria of interest and adapted to cause the bacteria of interest to release a reporter enzyme;

at least one first reagent control valve adapted to control a flow of the first reagent from the first reagent inlet port to the upstream end of the first filter chamber; and

a second filter chamber located downstream from the first filter chamber, the second filter chamber containing a second filter having a second area and formed from a second porous material adapted to specifically bind the reporter enzyme, wherein the second area is smaller than the first area; and

a detection chamber control valve located downstream of the first filter chamber and adapted to control a flow of fluid to the second filter chamber;

wherein the first filter is adapted to not bind the reporter enzyme.

Clause 2. The microfluidic device of clause 1, wherein the microfluidic device is adapted to process a fluid sample having a volume of at least about 100 ml.

Clause 3. The microfluidic device of clause 1, wherein two or more of the sample inlet port, the at least one reagent inlet port, the first filter chamber, and the second filter chamber are fluidically connected by at least one fluid channel having a width of about 2 mm and height of about 100 μm.

Clause 4. The microfluidic device of clause 1, wherein the first porous material includes at least one of polyvinyilidene fluoride (PVDF), polycarbonate (PC), tracked-etched polycarbonate (PCTE), polyethersulfone (PES), and tracked-etched polyester.

Clause 5. The microfluidic device of clause 1, wherein the first porous material has low protein binding activity.

Clause 6. The microfluidic device of clause 1, wherein the first porous material is a non-cellulose material.

Clause 7. The microfluidic device of clause 1, wherein the first porous material has a pore size of about 0.45 μm.

Clause 8. The microfluidic device of clause 1, wherein the first porous material has a pore size of less than about 0.45 μm.

Clause 9. The microfluidic device of clause 1, wherein the second porous material includes a cellulose-based material.

Clause 10. The microfluidic device of clause 1, wherein the second porous material includes at least one of regenerated cellulose, cellulose acetate, cellulose ester, and nitrocellulose.

Clause 11. The microfluidic device of clause 1, wherein the second porous material has a pore size of about 0.2 μm.

Clause 12. The microfluidic device of clause 1, wherein the second filter chamber includes a detection region configured to allow detection of a signal resulting from the reporter enzyme from outside the microfluidic device.

Clause 13. The microfluidic device of clause 1, wherein the first area is about 315 mm² and the second area is about 3.14 mm².

Clause 14. The microfluidic device of clause 1, wherein at least one of the sample control valve, the first reagent control valve, and detection chamber control valve includes a diaphragm valve.

Clause 15. The microfluidic device of clause 1, wherein at least one of the sample control valve, the first reagent control valve, and the detection chamber control valve includes a pneumatically controlled valve.

Clause 16. The microfluidic device of clause 15, including at least one air channel for connecting at least one pneumatic pressure source to the pneumatically controlled valve.

Clause 17. The microfluidic device of clause 1, including at least one air port fluidically connected to the upstream end of said first filter chamber and adapted for connection to a negative pressure source.

Clause 18. The microfluidic device of clause 1, including

at least one at least one waste port located downstream of the first filter chamber and adapted to receive fluid waste from the downstream end of the first filter chamber; and

at least one waste control valve adapted to control a flow of fluid waste from the downstream end of the first filter chamber to the at least one waste port.

Clause 19. The microfluidic device of clause 18, wherein the at least one waste port is adapted for connection to at least one negative pressure source.

Clause 20. The microfluidic device of clause 1, including

at least one at least one waste port located downstream of the second filter chamber and adapted to receive fluid waste from the downstream end of the second filter chamber.

Clause 21. The microfluidic device of clause 20, wherein the at least one waste port is adapted for connection to at least one negative pressure source.

Clause 22. The microfluidic device of clause 1, wherein the at least one first reagent inlet port is adapted to receive the first reagent from a reagent source.

Clause 23. The microfluidic device of clause 1, including a reservoir containing lyophilized reagent in fluid communication with the at least one reagent inlet port, wherein the at least one first reagent inlet port is adapted to receive a fluid adapted to rehydrate the lyophilized reagent to produce the first reagent for delivery to the first filter chamber.

Clause 24. The microfluidic device of clause 1, including

at least one second reagent inlet port located upstream of the first filter chamber and in fluid communication with the upstream end of the first filter chamber, the at least one said second reagent inlet port adapted to deliver to the first filter chamber a second reagent;

at least one second reagent control valve adapted to control a flow of the second reagent from the second reagent inlet port to the upstream end of the first filter chamber.

Clause 25. The microfluidic device of clause 24, including a reservoir containing lyophilized second reagent in fluid communication with the at least one second reagent inlet port, wherein the at least one second reagent inlet port is adapted to receive a fluid adapted to rehydrate the lyophilized second reagent to produce the second reagent for delivery to the first filter chamber.

Clause 26. The microfluidic device of clause 24, including

at least one third reagent inlet port located upstream of the first filter chamber and in fluid communication with the upstream end of the first filter chamber, the at least one said third reagent inlet port adapted to deliver to the first filter chamber a third reagent; and

at least one third reagent control valve adapted to control a flow of the third reagent from the third reagent inlet port to the upstream end of the first filter chamber.

Clause 27. The microfluidic device of clause 26, including a reservoir containing lyophilized third reagent in fluid communication with the at least one third reagent inlet port, wherein the at least one third reagent inlet port is adapted to receive a fluid capable of rehydrating the lyophilized third reagent to produce the third reagent for delivery to the first filter chamber.

Clause 28. The microfluidic device of clause 26, including

a bypass channel fluidically connecting the third reagent inlet port to the downstream end of the first filter chamber and the upstream end of the second filter chamber, and

a bypass valve adapted to control a flow of the third reagent from the third reagent inlet port to the downstream end of the first filter chamber and the upstream end of the second filter chamber.

Clause 29. The microfluidic device of clause 24, including

at least one third reagent inlet port in fluid communication with the downstream end of the first filter chamber and the upstream end of the second filter chamber, the at least one said third reagent inlet port adapted to deliver a third reagent to the second filter chamber; and

at least one third reagent control valve adapted to control a flow of the third reagent from the third reagent inlet port to the upstream end of the second filter chamber.

Clause 30. The microfluidic device of clause 1, formed from laminated polymeric sheet materials by a reel-to-reel process.

Clause 31. The microfluidic device of clause 1, formed from cast polymeric material.

Clause 32. The microfluidic device of clause 1, formed by injection molding.

Clause 33. A method of concentrating bacteria for detection, comprising:

introducing a fluid sample containing bacteria of interest in a carrier fluid to a sample inlet port of a microfluidic device;

drawing the carrier fluid through a first filter in a first filter chamber of the microfluidic device and through a waste port downstream of the first filter chamber while the bacteria of interest are captured by the first filter;

drawing a first reagent including growth media for the bacteria of interest from a first reagent inlet port into the first filter chamber;

incubating the bacteria of interest captured by the first filter with the first reagent in the first filter chamber for a first incubation period sufficient to increase at least one of the metabolic activity or the number of cells of the bacteria of interest;

drawing the first reagent through the first filter and through the waste port while the bacteria of interest remain captured by the first filter;

drawing a second reagent including a bacteriophage specific to the bacteria of interest from a second reagent inlet port into the first filter chamber;

incubating the bacteria of interest captured by the first filter with the second reagent in the first filter chamber for a second incubation period sufficient to produce expression of a reporter enzyme by the bacteria of interest;

drawing a fluid containing the expressed reporter enzyme through the first filter, through a second filter in a second filter chamber of the microfluidic device, and through the waste port while the expressed reporter enzyme is captured by the second filter; and

incubating the expressed reporter enzyme captured by the second filter with a third reagent in the second filter chamber for a third incubation period sufficient to produce a detectable signal in the detection chamber.

Clause 34. The method of clause 32, wherein the fluid containing the expressed reporter enzyme includes the third reagent, wherein the third reagent is drawn from a third reagent inlet port into the first filter chamber.

Clause 35. The method of clause 32, wherein the fluid containing the expressed reporter enzyme includes the second reagent, and wherein the third reagent is drawn from a third reagent inlet port into the second filter chamber.

Clause 36. The method of clause 32, including detecting the detectable signal with a luminometer.

Clause 37. The method of clause 32, wherein the fluid sample is a water sample.

Clause 38. The method of clause 32, wherein the bacteria of interest are Escherichia coli.

Clause 39. The method of clause 32, wherein the bacteria of interest are coliform bacteria.

Clause 40. The method of clause 32, wherein incubating the expressed reporter enzyme with the third reagent generates a chemiluminescent signal.

Clause 41. The method of clause 32, wherein incubating the expressed reporter enzyme with the third reagent generates a fluorescent signal.

Clause 42. The method of clause 32, wherein incubating the expressed reporter enzyme with the third reagent generates a colorimetric signal.

Clause 43. The method of clause 32, wherein the reporter enzyme has a cellulose-binding domain.

Clause 44. The method of clause 32, wherein the detectable signal corresponds to the amount of the expressed reporter enzyme captured by the second filter.

Clause 45. The method of clause 32, wherein the first incubation period lasts about 2 hours.

Clause 46. The method of clause 32, wherein the first incubation period lasts between about 1.5 hours and about 2.5 hours.

Clause 47. The method of clause 32, wherein the first incubation period is performed at about 37 degrees Celsius.

Clause 48. The method of clause 32, wherein the first incubation period is performed at between about 25 degrees Celsius and about 45 degrees Celsius.

Clause 49. The method of clause 32, wherein the second incubation period lasts about 1 hour.

Clause 50. The method of clause 32, wherein the second incubation period lasts between about 0.5 hours and about 2 hours.

Clause 51. The method of clause 32, wherein the second incubation period is performed at about 37 degrees Celsius.

Clause 52. The method of clause 32, wherein the second incubation period is performed at between about 25 degrees Celsius and about 45 degrees Celsius.

Clause 53. The method of clause 32, wherein drawing the carrier fluid through the first filter in the first filter chamber of the microfluidic device and through the waste port downstream of the first filter chamber while the bacteria of interest are captured by the first filter includes opening a sample control valve between the sample inlet port and the filter chamber, opening a waste control valve downstream of the filter chamber, and applying a negative pressure at the waste port downstream of the filter chamber.

Clause 54. The method of clause 32, including drawing at least one of the first reagent, the second reagent, and the third reagent through the waste port and into a waste reservoir.

Clause 55. The method of clause 32, including drawing at least one of the first reagent, the second reagent, and the third reagent through the waste port and into a reagent reservoir.

Clause 56. The method of clause 32, wherein drawing the first reagent including growth media for the bacteria of interest from the first reagent inlet port into the filter chamber includes closing the sample control valve and waste control valve, opening a first reagent control valve between the first reagent inlet port and the filter chamber, opening a vent control valve between the filter chamber and a vent outlet, and applying a negative pressure to the vent outlet.

Clause 57. The method of clause 32, wherein the first reagent includes Luria-Bertani media.

Clause 58. The method of clause 32, wherein incubating the bacteria of interest captured by the first filter with the first reagent in the filter chamber for the first incubation period sufficient to increase at least one of the metabolic activity or the number of cells of the bacteria of interest includes closing a first reagent control valve and a vent control valve.

Clause 59. The method of clause 32, wherein drawing the first reagent through the first filter and through the waste port while the bacteria of interest remain captured by the first filter includes opening a vent control valve and a waste control valve and applying a negative pressure at the waste port.

Clause 60. The method of clause 32, wherein the bacteriophage includes an engineered reporter bacteriophage.

Clause 61. The method of clause 32, wherein the bacteriophage includes a reporter bacteriophage specific to the bacteria of interest.

Clause 62. The method of clause 32, wherein the bacteriophage is adapted to lyse the bacteria of interest to release a reporter enzyme.

Clause 63. The method of clause 32, wherein the second reagent includes a fluid containing a cocktail of reporter bacteriophages.

Clause 64. The method of clause 32, wherein the second reagent includes a fluid containing a reporter enzyme.

Clause 65. The method of clause 32, wherein the second reagent includes T7-NanoLuc®-Cellulose Binding Module.

Clause 66. The method of clause 32, wherein drawing the second reagent including the bacteriophage specific to the bacteria of interest from the second reagent inlet port into the first filter chamber includes closing a waste control valve, opening a second reagent control valve between the second reagent inlet port and the first filter chamber, and applying a negative pressure to the vent outlet.

Clause 67. The method of clause 32, wherein incubating the bacteria of interest captured by the first filter with the second reagent in the first filter chamber includes closing a second reagent control valve and a vent control valve.

Clause 68. The method of clause 33, wherein drawing the fluid containing the expressed reporter enzyme through the first filter, through the second filter in the second filter chamber of the microfluidic device, and through the waste port while the expressed reporter enzyme is captured by the second filter includes opening a third reagent control valve between a third reagent inlet port and the first filter chamber, opening a detection chamber control valve downstream of the first filter chamber, and applying a negative pressure at the waste port, wherein the second filter chamber is fluidically connected between the detection chamber control valve and the waste port.

Clause 69. The method of clause 32, wherein incubating the expressed reporter enzyme captured by the second filter with the third reagent in the second filter chamber for the third incubation period includes closing the third reagent control valve and the detection chamber control valve.

Clause 70. The method of clause 34, including

drawing the fluid containing the expressed reporter enzyme through the first filter, through the second filter in the second filter chamber of the microfluidic device, and through the waste port while the expressed reporter enzyme is captured by the second filter by opening a vent control valve upstream of the first filter chamber, opening a detection chamber control valve fluidically connected between the downstream end of the first filter chamber and an upstream end of the second filter chamber and applying a negative pressure at the waste port, wherein the second filter chamber is fluidically connected between the detection chamber control valve and the waste port; and

drawing the third reagent into the second filter chamber prior to the third incubation period by closing the vent upstream of the first filter chamber, opening a third reagent control valve fluidically connected between a third reagent inlet port and a downstream end of the first filter chamber, opening a detection chamber control valve, and applying a negative pressure at the waste port.

Clause 71. A microfluidic device for bacteria detection, comprising:

a sample inlet port for receiving a fluid sample containing bacteria of interest;

a first filter chamber containing a first filter adapted for capturing bacteria of interest from the fluid sample;

first microfluidic means for introducing bacterial growth media to the first filter chamber;

second microfluidic means for introducing phage specific to the bacteria of interest to the first filter chamber, the phage adapted to cause the bacteria of interest to produce a reactive material capable of reacting to produce a detectable signal;

third microfluidic means for flushing reactive material from the first filter chamber, the reactive material released from the bacteria of interest responsive to introduction of the phage; and

a second filter chamber containing a second filter for specifically capturing the reactive material flushed from the first filter chamber, wherein the second filter is smaller than the first filter to amplify the detectable signal;

wherein the first filter is adapted to not capture the reactive material.

Clause 72. The microfluidic device of clause 70, including lysing means for lysing the bacteria of interest to release the reactive material.

Clause 73. The microfluidic device of clause 71, wherein the lysing means includes heating means.

Clause 74. The microfluidic device of clause 71, wherein the lysing means includes acoustic means.

Clause 75. The microfluidic device of clause 71, wherein the lysing means includes a pressure source.

Clause 76. The microfluidic device of clause 71, wherein the lysing means includes a reagent source.

Clause 77. The microfluidic device of clause 71, wherein the lysing means includes an enzyme source.

Clause 78. The microfluidic device of clause 71, wherein at least one of the first microfluidic means, the second microfluidic means, and the third microfluidic means includes a reservoir containing lyophilized reagent.

Clause 79. The microfluidic device of clause 71, wherein at least one of the first microfluidic means, the second microfluidic means, and the third microfluidic means includes a port for interfacing with an external fluid source.

Clause 80. The microfluidic device of clause 71, wherein at least one of the first microfluidic means, the second microfluidic means, and the third microfluidic means includes at least one microchannel and at least one valve.

Clause 81. The microfluidic device of clause 79, wherein the at least one valve includes at least one pneumatically actuated valve and at least one air channel adapted for connection to a pressure source.

Clause 82. The microfluidic device of clause 79, including at least one negative pressure source located downstream of the at least one valve.

Clause 83. The microfluidic device of clause 81, wherein the at least one negative pressure source is located downstream of first filter chamber.

Clause 84. The microfluidic device of clause 70, wherein the first filter includes a porous non-cellulose material having a pore size of about 0.45 μm, and wherein the second filter includes a cellulose-based material.

Clause 85. The microfluidic device of clause 70, wherein the second filter chamber includes a detection region configured to allow detection of the detectable signal from outside the microfluidic device.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

The herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A microfluidic device comprising:
a sample inlet port adapted to receive a fluid sample containing bacteria of interest;
a first filter chamber located downstream from the sample inlet port, the first filter chamber containing a first filter having a first area and formed from a first porous material having a pore size adapted to capture the bacteria of interest;
a sample inlet channel connecting the sample inlet port to an upstream end of the first filter chamber;
a sample control valve in the sample inlet channel, the sample control valve adapted to control a flow of the sample fluid from the sample inlet port to the upstream end of the first filter chamber;
at least one first reagent inlet port located upstream of the first filter chamber and in fluid communication with the upstream end of the first filter chamber, the at least one first reagent inlet port adapted to deliver to the first filter chamber a first reagent containing a bacteriophage specific to the bacteria of interest and adapted to cause the bacteria of interest to release a reporter enzyme;
at least one first reagent control valve adapted to control a flow of the first reagent from the first reagent inlet port to the upstream end of the first filter chamber; and
a second filter chamber located downstream from the first filter chamber, the second filter chamber containing a second filter having a second area and formed from a second porous material adapted to specifically bind the reporter enzyme, wherein the second area is smaller than the first area; and
a detection chamber control valve located downstream of the first filter chamber and adapted to control a flow of fluid to the second filter chamber;
wherein the first filter is adapted to not bind the reporter enzyme.

2. The microfluidic device of claim 1, wherein the microfluidic device is adapted to process a fluid sample having a volume of at least about 100 ml.

3.-11. (canceled)

12. The microfluidic device of claim 1, wherein the second filter chamber includes a detection region configured to allow detection of a signal resulting from the reporter enzyme from outside the microfluidic device.

13.-17. (canceled)

18. The microfluidic device of claim 1, including
at least one at least one waste port located downstream of the first filter chamber and adapted to receive fluid waste from the downstream end of the first filter chamber; and
at least one waste control valve adapted to control a flow of fluid waste from the downstream end of the first filter chamber to the at least one waste port.

19. (canceled)

20. The microfluidic device of claim 1, including
at least one at least one waste port located downstream of the second filter chamber and adapted to receive fluid waste from the downstream end of the second filter chamber.

21.-32. (canceled)

33. A method of concentrating bacteria for detection, comprising:
introducing a fluid sample containing bacteria of interest in a carrier fluid to a sample inlet port of a microfluidic device;
drawing the carrier fluid through a first filter in a first filter chamber of the microfluidic device and through a waste port downstream of the first filter chamber while the bacteria of interest are captured by the first filter;
drawing a first reagent including growth media for the bacteria of interest from a first reagent inlet port into the first filter chamber;
incubating the bacteria of interest captured by the first filter with the first reagent in the first filter chamber for a first incubation period sufficient to increase at least one of the metabolic activity or the number of cells of the bacteria of interest;
drawing the first reagent through the first filter and through the waste port while the bacteria of interest remain captured by the first filter;
drawing a second reagent including a bacteriophage specific to the bacteria of interest from a second reagent inlet port into the first filter chamber;
incubating the bacteria of interest captured by the first filter with the second reagent in the first filter chamber for a second incubation period sufficient to produce expression of a reporter enzyme by the bacteria of interest;
drawing a fluid containing the expressed reporter enzyme through the first filter, through a second filter in a second filter chamber of the microfluidic device, and through the waste port while the expressed reporter enzyme is captured by the second filter; and
incubating the expressed reporter enzyme captured by the second filter with a third reagent in the second filter chamber for a third incubation period sufficient to produce a detectable signal in the detection chamber.

34. The method of claim 33, wherein the fluid containing the expressed reporter enzyme includes the third reagent, wherein the third reagent is drawn from a third reagent inlet port into the first filter chamber.

35. The method of claim 33, wherein the fluid containing the expressed reporter enzyme includes the second reagent, and wherein the third reagent is drawn from a third reagent inlet port into the second filter chamber.

36. The method of claim 33, including detecting the detectable signal with a luminometer.

37. The method of claim 33, wherein the fluid sample is a water sample.

38.-42. (canceled)

43. The method of claim 33, wherein the reporter enzyme has a cellulose-binding domain.

44. The method of claim 33, wherein the detectable signal corresponds to the amount of the expressed reporter enzyme captured by the second filter.

45.-52. (canceled)

53. The method of claim 33, wherein drawing the carrier fluid through the first filter in the first filter chamber of the microfluidic device and through the waste port downstream of the first filter chamber while the bacteria of interest are captured by the first filter includes opening a sample control valve between the sample inlet port and the filter chamber, opening a waste control valve downstream of the filter chamber, and applying a negative pressure at the waste port downstream of the filter chamber.

54.-55. (canceled)

56. The method of claim 33, wherein drawing the first reagent including growth media for the bacteria of interest from the first reagent inlet port into the filter chamber includes closing the sample control valve and waste control valve, opening a first reagent control valve between the first reagent inlet port and the filter chamber, opening a vent control valve between the filter chamber and a vent outlet, and applying a negative pressure to the vent outlet.

57. (canceled)

58. The method of claim 33, wherein incubating the bacteria of interest captured by the first filter with the first reagent in the filter chamber for the first incubation period sufficient to increase at least one of the metabolic activity or the number of cells of the bacteria of interest includes closing a first reagent control valve and a vent control valve.

59. The method of claim 33, wherein drawing the first reagent through the first filter and through the waste port while the bacteria of interest remain captured by the first filter includes opening a vent control valve and a waste control valve and applying a negative pressure at the waste port.

60.-65. (canceled)

66. The method of claim 33, wherein drawing the second reagent including the bacteriophage specific to the bacteria of interest from the second reagent inlet port into the first filter chamber includes closing a waste control valve, opening a second reagent control valve between the second reagent inlet port and the first filter chamber, and applying a negative pressure to the vent outlet.

67. The method of claim 33, wherein incubating the bacteria of interest captured by the first filter with the second reagent in the first filter chamber includes closing a second reagent control valve and a vent control valve.

68. The method of claim 34, wherein drawing the fluid containing the expressed reporter enzyme through the first filter, through the second filter in the second filter chamber of the microfluidic device, and through the waste port while the expressed reporter enzyme is captured by the second filter includes opening a third reagent control valve between a third reagent inlet port and the first filter chamber, opening a detection chamber control valve downstream of the first filter chamber, and applying a negative pressure at the waste port, wherein the second filter chamber is fluidically connected between the detection chamber control valve and the waste port.

69. The method of claim 33, wherein incubating the expressed reporter enzyme captured by the second filter with the third reagent in the second filter chamber for the third incubation period includes closing the third reagent control valve and the detection chamber control valve.

70. The method of claim 35, including
drawing the fluid containing the expressed reporter enzyme through the first filter, through the second filter in the second filter chamber of the microfluidic device, and through the waste port while the expressed reporter enzyme is captured by the second filter by opening a vent control valve upstream of the first filter chamber, opening a detection chamber control valve fluidically connected between the downstream end of the first filter chamber and an upstream end of the second filter chamber and applying a negative pressure at the waste port, wherein the second filter chamber is fluidically connected between the detection chamber control valve and the waste port; and
drawing the third reagent into the second filter chamber prior to the third incubation period by closing the vent upstream of the first filter chamber, opening a third reagent control valve fluidically connected between a third reagent inlet port and a downstream end of the first filter chamber, opening a detection chamber control valve, and applying a negative pressure at the waste port.

71. A microfluidic device for bacteria detection, comprising:
a sample inlet port for receiving a fluid sample containing bacteria of interest;
a first filter chamber containing a first filter adapted for capturing bacteria of interest from the fluid sample;
first microfluidic means for introducing bacterial growth media to the first filter chamber;
second microfluidic means for introducing phage specific to the bacteria of interest to the first filter chamber, the phage adapted to cause the bacteria of interest to produce a reactive material capable of reacting to produce a detectable signal;
third microfluidic means for flushing reactive material from the first filter chamber, the reactive material released from the bacteria of interest responsive to introduction of the phage; and
a second filter chamber containing a second filter for specifically capturing the reactive material flushed from the first filter chamber, wherein the second filter is smaller than the first filter to amplify the detectable signal;
wherein the first filter is adapted to not capture the reactive material.

72. The microfluidic device of claim 71, including lysing means for lysing the bacteria of interest to release the reactive material.

73.-79. (canceled)

80. The microfluidic device of claim 72, wherein at least one of the first microfluidic means, the second microfluidic means, and the third microfluidic means includes at least one microchannel and at least one valve.

81.-83. (canceled)

84. The microfluidic device of claim 71, wherein the first filter includes a porous non-cellulose material having a pore size of about 0.45 μm, and wherein the second filter includes a cellulose-based material.

85. The microfluidic device of claim 71, wherein the second filter chamber includes a detection region configured to allow detection of the detectable signal from outside the microfluidic device.

86. The microfluidic device of claim 1, wherein the first porous material includes at least one of polyvinyilidene fluoride (PVDF), polycarbonate (PC), tracked-etched polycarbonate (PCTE), polyethersulfone (PES), and tracked-etched polyester, a material having low protein binding activity, a non-cellulose material, and a material having a pore size of about 0.45 μm.

87. The microfluidic device of claim 1, wherein the second porous material includes at least one of a cellulose-based material, regenerated cellulose, cellulose acetate, cellulose ester, nitrocellulose, and a material having a pore size of about 0.2 μm.

88. The microfluidic device of claim 1, wherein at least one of the sample control valve, the first reagent control valve, and detection chamber control valve includes a diaphragm valve or a pneumatically controlled valve.

89. The microfluidic device of claim 1, wherein the at least one first reagent inlet port is adapted to receive the first reagent from at least one of a reagent source, and a reservoir containing lyophilized reagent in fluid communication with the at least one reagent inlet port, wherein the at least one first reagent inlet port is adapted to receive a fluid adapted to rehydrate the lyophilized reagent to produce the first reagent for delivery to the first filter chamber.

90. The microfluidic device of claim 1, including at least one of:
at least one second reagent inlet port located upstream of the first filter chamber and in fluid communication with the upstream end of the first filter chamber, the at least one said second reagent inlet port adapted to deliver to the first filter chamber a second reagent, and at least one second reagent control valve adapted to control a flow of the second reagent from the second reagent inlet port to the upstream end of the first filter chamber;
at least one third reagent inlet port located upstream of the first filter chamber and in fluid communication with the upstream end of the first filter chamber, the at least one said third reagent inlet port adapted to deliver to the first filter chamber a third reagent, and at least one third reagent control valve adapted to control a flow of the third reagent from the third reagent inlet port to the upstream end of the first filter chamber; and
at least one third reagent inlet port in fluid communication with the downstream end of the first filter chamber and the upstream end of the second filter chamber, the at least one said third reagent inlet port adapted to deliver a third reagent to the second filter chamber, and at least one third reagent control valve adapted to control a flow of the third reagent from the third reagent inlet port to the upstream end of the second filter chamber.

91. The method of claim 33, wherein the bacteria of interest include at least one of Escherichia coli and coliform bacteria.

92. The method of claim 33, wherein incubating the expressed reporter enzyme with the third reagent generates at least one of a chemiluminescent signal, a fluorescent signal, and a colorimetric signal.

93. The method of claim 33, wherein the first incubation period lasts between about 1.5 hours and about 2.5 hours and is performed at between about 25 degrees Celsius and about 45 degrees Celsius.

94. The method of claim 33, wherein the second incubation period lasts between about 0.5 hours and about 2 hours and is performed at between about 25 degrees Celsius and about 45 degrees Celsius.

95. The method of claim 33, including at least one of:
drawing at least one of the first reagent, the second reagent, and the third reagent through the waste port and into a waste reservoir; and
drawing at least one of the first reagent, the second reagent, and the third reagent through the waste port and into a reagent reservoir.

96. The method of claim 33, wherein the bacteriophage includes at least one of an engineered reporter bacteriophage, a reporter bacteriophage specific to the bacteria of interest, and a bacteriophage adapted to lyse the bacteria of interest to release a reporter enzyme.

97. The method of claim 33, wherein the second reagent includes at least one of a fluid containing a cocktail of reporter bacteriophages, a fluid containing a reporter enzyme, and T7-NanoLuc®-Cellulose Binding Module.

98. The microfluidic device of claim 72, wherein the lysing means includes at least one of heating means, acoustic means, a pressure source, a reagent source, and an enzyme source.

99. The microfluidic device of claim 72, wherein at least one of the first microfluidic means, the second microfluidic means, and the third microfluidic means includes a reservoir containing lyophilized reagent or a port for interfacing with an external fluid source.
